(19) United States
(12) Patent Application Publication
(10) Pub. No.: US 2002/0035070 A1
(43) Pub. Date: Mar. 21, 2002

(54) Method of regulating hair growth using metal complexes of oxidized carbohydrates

(75) Inventors: John Michael Gardlik, Cincinnati, OH (US); Diana Severynse-Stevens, Yardley, PA (US); Bryan Gabriel Comstock, Mason, OH (US)

Correspondence Address: Brent M. Peebles, The Procter & Gamble Company, Sharon Woods Technical Center, 11511 Reed Hartman Highway, Cincinnati, OH, 45241, US

(73) Assignee: The Procter & Gamble Company

(21) Appl. No.: 09/909,441

(22) Filed: Jul. 19, 2001

Related U.S. Application Data
(60) Provisional application No. 60/220,755, filed on Jul. 26, 2000.

(51) Int. Cl. A61K 31/70; A61K 31/315

(52) U.S. Cl. 514/23; 514/492; 514/495; 514/499; 514/494

(57) ABSTRACT

A method for regulating the growth of hair comprising administering to a mammal, an effective amount of a composition comprising: (a) from about 0.001% to about 99.9%, by weight, of at least one metal complex of an oxidized carbohydrate, wherein the metal complex of an oxidized carbohydrate is neither zinc gluconate nor manganese gluconate; and (b) from about 0.1% to about 99.999%, by weight, of a vehicle.

### TECHNICAL FIELD OF THE INVENTION

The present invention relates to a method of regulating hair growth comprising the administering, preferably topically, of compositions containing: (a) metal complexes of oxidized carbohydrates, but neither zinc gluconate nor manganese gluconate; and (b) a vehicle.

### BACKGROUND OF THE INVENTION

The hair of a mammal has always been recognized as an important symbol of beauty, strength, vitality and fashion, whether it be a lion's mane, a dog's shiny coat, or a human's locks. The immense strength of the great Samson was reputedly drained as Delilah severed his hair. The Egyptian queen Cleopatra was known to immerse herself in luxuriant baths of milk and honey to revitalize her hair and skin. Upon the emergence of post-medieval English and French courts, those charged with administering the law were distinguished by wearing elaborate wigs.

In contemporary times, the icon of an adult male lion with a brandishing mane is associated with pride, strength, and tradition. Prizes are awarded at shows for dogs having healthy, shiny coats. Humans are willing to invest much time and money to maintain their “do,” for sundry reasons, including making a social or business appearance, “expressing” oneself, demonstrating political speech, being attractive, and being healthy. However, many do not enjoy a full amount of hair. Generally, the absence of hair from areas where it normally grows is referred to as “alopecia.”

Because such importance has been placed on the quantity and quality of hair, as described above, those who suffer from some form of alopecia are often stigmatized. These men, women, and children often experience a self-consciousness related to their condition, and may feel an emotional trauma and lack of self-esteem. While perhaps issued in good humor, bald jokes manufactured by would-be comedians are sometimes received by the hair loss sufferer as injurious.

In most mammals, hair does not grow continuously, but rather, it undergoes a cycle of activity involving alternate periods of growth and rest. The hair growth cycle can typically be divided into three main stages, namely: (a) the growth phase known as anagen, during which the hair follicle penetrates deep into the dermis with the cells of the bulb dividing rapidly and differentiating to form the hair; (b) the transitional stage known as catagen, which is heralded by the cessation of mitosis, and during which the follicle regresses upwards through the dermis and hair growth ceases; and (c) the resting stage known as telogen, in which the regressed follicle contains a small secondary germ with an underlying ball of tightly packed dermal papilla cells. The initiation of a new anagen phase is revealed by rapid proliferation in the germ, expansion of the dermal papilla and elaboration of basement membrane components.

Alopecia may be initiated or aggravated by many factors or conditions. Alopecia may be hereditary, as in the case of male pattern baldness, or it may be due to disease, such as malnutrition, injury or insult, such as overdrying, bleaching, coloring, overbrushing, or treatment, such as chemotherapy, or it may occur in old age, or it may be due to physical, psychological or emotional stress, or still yet, it may be caused by hormonal imbalance, particularly during the advent of menopause. The rate of hair growth may decrease, the rate of hair loss may increase, or the structure, including thickness of the hair may be altered. Also, hair may fall out gradually, or in patches.

It is believed that androgenetic alopecia (commonly known as “male pattern baldness” or “female pattern baldness”) is the result of hyperandrogenic stimulation caused by excessive accumulation of testosterone or similar androgenic hormones in the metabolic system. A principal mediator of androgenic activity is dihydrotestosterone (DHT), formed locally in the target area by the action of 5-α-reductase. Inhibitors of this enzyme will serve to diminish symptoms of hyperandrogenic stimulation in these target areas. The enzyme 5-α-reductase catalyzes the reduction of testosterone to the more potent androgen, DHT, as shown below:

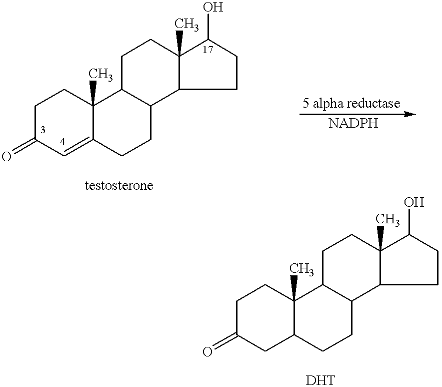

Another mechanism believed to contribute to alopecia is inflammation of the hair follicle. During the inflammatory process, cytokines (e.g. interleukin-1-α, interleukin-1-β, and tumor necrosis factor) are released that have been demonstrated to inhibit hair growth. Antibodies produced by the immune system may also play a role in inhibiting hair growth. Accordingly, agents which inhibit inflammation, i.e. anti-inflammatories, may be used to regulate hair growth. Also, compounds that inhibit the immune system's ability to attach leukocytes to the follicle may be useful in regulating hair growth.

In response to consumer interest in combating alopecia, many products and methods have been advertised as being capable of providing benefits ranging from cosmetic masking of alopecia to “curing” hair loss. There are many internet websites which are dedicated to providing information on alopecia, which enable the purchase of products that are believed to combat alopecia, or which at least provides hyperlinks to other websites of these types. Various regimens are available for combating alopecia: (a) topical treatments for growing hair or for retarding the loss of hair (such as ROGAINE (RTM), available from Pharmacia & Upjohn); (b) oral treatments for growing hair or for retarding the loss of hair (such as PROPECIA (RTM), available from Merck Pharmaceuticals); (c) shampoos for making hair appear thicker and fuller (such as COUVRE (TM) Thickening Shampoo, available from Spencer Forrest, Inc.); (d) hair loss concealers for creating a cosmetic appearance that hair is not as thin as it really is (such as PROTHIK (TM) Spray, available from Aquila); (e) artificial hairpieces (such as toupees or weaves); (f) and surgical transplanting. While these regimens may partially address alopecia for certain individuals, they have various limitations. For instance, not all people respond to ROGAINE (RTM) (active ingredient is minoxidil (6-(1-piperidinyl)-2,4-pyrimidinediamine 3-oxide)) (see U.S. Pat. Nos. 3,461,461; 3,973,061; 3,464,987; and 4,139,619), and the efficacy level is limited in those who do exhibit a response. Many people find ROGAINE (RTM) to be expensive, and its side effects include itching, scaling and scalp irritation. Thickening shampoos and concealers do not actually increase hair growth or retard hair loss. Rather, these regimens merely cosmetically mask hair loss, oftentimes by coloring the scalp to reduce the contrast between hair and scalp. Many products of this type are ineffective in providing a natural look, may be difficult to remove, and may run or dilute when exposed to moisture. Artificial hair pieces may come unattached causing an embarrassing moment, and oftentimes fail to provide a natural look. Surgical transplanting of “hair plugs,” vulgarly known as “sodding,” may be very time consuming, expensive, painful, and during the period after the surgery may appear unattractive. Recently, there has been an explosion of new products on the market which claim to be inexpensive, all-natural, and are supported by one or two testimonials of persons who believe to have seen an increase in hair growth. Such products are usually comprised of plant extracts, vitamins, amino acids, plant proteins, herbs, plant oils, and berries. Many of these products are speculative and have yet to be proven clinically.

PROPECIA (RTM) (finasteride) is a synthetic 4-azasteroid compound, that is a specific inhibitor of steroid Type II 5-α-reductase, an intracellular enzyme that converts the androgen testosterone into 5-α-dihydrotestosterone (DHT). A reduction in DHT level is believed to correlate to a reduction in alopecia. Finasteride is 4-azaandrost-1-ene-17-carboxamide,N-(1,1-dimethylethyl)-3-oxo-,(5α,17β)—, (see U.S. Pat. No. 5,670,643; EP 823,436; WO 97/15558; and WO 97/15564). There are a limited number of people which respond to PROPECIA (RTM), and its efficacy level is limited in those who exhibit a response. Also, many people find PROPECIA (RTM) to be expensive. It is for use by men only, and due to the severe risk of teratogenic effects, the manufacturer warns that women who are or who may potentially be pregnant should not even handle broken pills. The manufacturer also reports that in clinical studies a small number of men experienced certain sexual side effects.

Accordingly, there exists a need for a method for regulating the growth of hair which appeals to a larger number of consumers, at a reasonable price, which provides good results, with few, if any, undesirable side effects. Applicants have found, surprisingly, that by applying compositions containing certain metal complexes of oxidized carbohydrates, the growth of hair in mammals can be regulated, and as such, alopecia can be combated. It is believed that zinc and other metals may regulate hair growth by (a) inhibiting activity of 5-α-reductase which converts testosterone to DHT, and (b) inhibiting DHT binding to the androgen receptor in the cytosol. Zinc and other metals may also have an anti-inflammatory effect on hair follicles, which is believed to correlate to a reduction in alopecia. Applicants have discovered that certain metal complexes of oxidized carbohydrates can (a) disrupt DHT activity; and/or (b) stimulate the transition of follicles from the resting telogen phase into the active anagen phase and/or from earlier anagen phase to later anagen phase; and/or (c) retard the transition of follicles from anagen phase to catagen phase; and/or (d) may also have an anti-inflammatory effect on hair follicles.

### SUMMARY OF THE INVENTION

The present invention relates to a method for regulating the growth of hair comprising administering to a mammal, an effective amount of a composition comprising: (a) from about 0.001% to about 99.9%, by weight, of at least one metal complex of an oxidized carbohydrate, wherein the metal complex of an oxidized carbohydrate is neither zinc gluconate nor manganese gluconate; and (b) from about 0.1% to about 99.999%, by weight, of a vehicle.

### DETAILED DESCRIPTION OF THE INVENTION

The present invention relates to a method for regulating the growth of hair. Particularly, these compositions comprise (a) at least one metal complex of an oxidized carbohydrate, but neither zinc gluconate nor manganese gluconate; and (b) a vehicle. These compositions, when applied to a mammal, may disrupt DHT activity, stimulate the transition of follicles from the resting telogen phase into the active anagen phase, and may, in some cases, also have an anti-inflammatory effect. These characteristics are important to regulating the growth of hair.

Such compositions of the present invention can comprise, consist of, or consist essentially of the essential elements and limitations of the invention described herein, as well as any of the additional or optional ingredients, components or limitations described herein. The components of the compositions of the present invention, including those which may optionally be added, as well as methods for preparation, and methods for use thereof, are described in detail below.

As used herein the term “hair” is meant to encompass all mammalian hair. Preferably, the hair is that of a human, however, animal hair, such as dog hair and cat hair are suitable. While the hair whose growth is to be regulated is typically located upon the head, it is contemplated that the inventive method and compositions described herein may be applied to hair located anywhere on the body, including, eyebrows, mustaches, beards, the pubic area, and anywhere else the consumer prefers.

As used herein, the terms “regulating hair growth,” “hair growth regulation,” and “regulating the growth of hair,” are meant to include: stimulating hair growth; stimulating hair thickening; preventing, reducing, arresting and/or retarding the loss of hair; preventing, reducing, arresting and/or retarding the thinning of hair; increasing the rate of hair growth; inducing the formation of a greater number of hair strands; increasing the diameter of the hair strand; lengthening the hair strand; changing the hair follicle from vellus follicle to terminal follicle; inducing the formation of vellus follicles; converting follicles from telogen to anagen phase (thereby increasing the overall ratio of anagen phase follicles relative to telogen phase follicles); advancing a follicle from an earlier stage of anagen to a later stage of anagen; reducing the conversion from anagen to catagen phase; treating alopecia; and any combination thereof.

As used herein, the term “vellus follicle” means a hair follicle which produces a soft, short, and often colorless hair fiber. The size of the vellus follicle is considerably smaller than the terminal hair follicle. In an adult, vellus follicles can be found on the forehead (i.e., receding hair line area) and bald scalp.

As used herein, the term “terminal follicle” means a hair follicle which produces a coarse, long and often pigmented hair shaft. The size of the terminal follicle is considerably larger and thicker in diameter and longer than the vellus follicle. In an adult, terminal follicles can be found on the scalp, axilla and pubic areas.

As used herein, “anagen phase” refers to the period in the hair follicle growth cycle wherein the follicle is actively growing and producing new hair.

As used herein, “telogen phase” refers to the period in the hair growth cycle wherein the follicle is resting and not producing new hair.

As used herein, the term “oxidized carbohydrate” is meant to be inclusive of acids derived from carbohydrates. The adjective “oxidized” is meant to be inclusive of mono-, di-, and poly-oxidized. The term “carbohydrate” is meant to be inclusive of mono-, di-, oligo-, and poly-saccharides.

The term “safe and effective amount” as used herein, means an amount of an active ingredient high enough to modify the condition to be treated or to deliver the desired hair growth regulation benefit, but low enough to avoid serious side effects, at a reasonable benefit to risk ratio within the scope of sound medical judgment. What is a safe and effective amount of the active ingredient will vary with the specific active, the ability of the active to penetrate through the skin, hair, or relevant tissue of the digestive tract, the age, health condition, and skin, hair or digestive condition of the user, and other like factors.

### I. Components

The method of the present invention utilizes compositions which comprise at least one metal complex of an oxidized carbohydrate and a vehicle. Such compositions can be administered topically, orally or parenterally, preferably topically. Such compositions can be in any form which delivers a sufficient amount of the metal complex of an oxidized carbohydrate to effectively regulate hair growth. Such forms include, but are not limited to tablets, capsules, caplets, creams, gels, hydrogels, lotions, shampoos, rinses, tonics, sprays, ointments, mousses or pomade.

The ingredients comprising the compositions herein, as well as other optional components, are described in detail as follows. As is known in the art, many cosmetic ingredients have multiple functions in formulations and therefore may be included in several functional groupings. Accordingly, it should be understood that although the active ingredients useful herein are categorized by their therapeutic benefit or their postulated mode of action, some such ingredients can in some instances provide more than one cosmetic and/or therapeutic benefit or operate via more than one mode of action. Therefore, classifications herein are made for the sake of convenience and are not intended to limit the active ingredient to that particular application or applications listed. Also, where not stated otherwise, cosmetically and pharmaceutically acceptable salts of these active ingredients are useful herein.

It is to be understood that the percentage weights of the composition components herein are expressed in terms of the total composition, and includes the composition in the form of intended use.

A. Metal Complex of an Oxidized Carbohydrate

The method of the present invention utilizes compositions which comprise as an essential component, at least one metal complex of an oxidized carbohydrate in an amount which is sufficiently effective to regulate hair growth, wherein the at least one metal complex of an oxidized carbohydrate is neither zinc gluconate nor manganese gluconate. As described below, however, zinc gluconate and manganese gluconate may optionally be included as additional ingredients. Typically an amount effective to regulate hair growth will range from about 0.001% to about 99.9%, by weight of the composition, preferably from about 0.001% to about 75%, more preferably from about 0.001% to about 50%, more preferably from about 0.001% to about 25%, more preferably from about 0.001% to about 15%.

The metal complexes of oxidized carbohydrates are believed to: (a) disrupt DHT activity; and/or (b) stimulate the transition of follicles from the resting telogen phase into the active anagen phase and/or from earlier anagen phase to later anagen phase; and/or (c) retard the transition of follicles from anagen phase to catagen phase; and/or (d) may also have an anti-inflammatory effect on hair follicles. These characteristics are important to regulating hair growth.

The metal complexes of oxidized carbohydrates, are the result of an equilibrium reached between metal ions, and deprotonated carboxylic acids of carbohydrates, as shown, without regard to stereochemistry, in formulae (I) and (II), below. This equilibrium may be achieved during or before, preferably before, administration of the composition according to the method described herein.

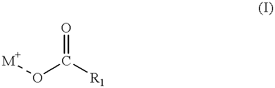

wherein M is a monovalent metal ion, and

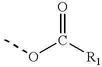

is an oxidized carbohydrate, R₁ being the remainder of the carbohydrate moiety;

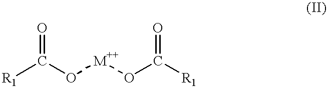

wherein M is a bivalent metal ion, and

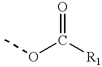

is an oxidized carbohydrate, R₁ being the remainder of the carbohydrate moiety;

The metal complex of an oxidized carbohydrate component of the compositions herein may be prepared by any suitable means. For example: metal-salt + carboxylic acid of carbohydrate yields metal complex of an oxidized carbohydrate and the conjugate acid of the metal's counter ion. The pH of the resulting product may be adjusted using any suitable pH adjuster. A non-limiting example of this preparation is: zinc sulfate +lactobionic acid yields zinc lactobionate and sulphuric acid, with the pH being adjusted to the desired level with sodium hydroxide. Other non-limiting examples may be constructed using the metals, salts, oxidized carbohydrates, and pH adjusters described as suitable in the disclosure herein. Typically, from about 0.5% to about 50%, preferably about 1% to about 25%, more preferably from about 3% to about 10%, of metal-salt will be added to typically about 0.5% to about 50%, preferably from about 1% to about 30%, more preferably from about 5% to about 25%, of carboxylic acid.

Suitable salts for use herein include, but are not limited to: the chlorides, sulfates, acetates, or oxides of sodium, lithium, potassium, silver, gold, zinc, copper, nickel, iron, chromium, calcium, magnesium, molybdenum, manganese, cobalt, palladium, platinum, and tin. Non-limiting examples of preferred salts are zinc sulfate, zinc acetate, zinc oxide, cupric chloride, cupric sulfate, cupric acetate, and copper oxide.

1. Monovalent Metal

The metal complex of an oxidized carbohydrate component of the compositions herein comprises metals. Such metals may be in an oxidation state or valence of 1+. Monovalent metals which are suitable for use herein include: lithium; silver; gold; sodium; and mixtures thereof. Preferred metals are sodium and lithium.

2. Bivalent Metal

The metal complex of an oxidized carbohydrate component of the compositions herein comprises metals. Such metals may preferably be in an oxidation state or valence of 2+. Bivalent metals which are suitable for use herein include: zinc, copper, nickel, iron, chromium, calcium, magnesium, molybdenum, manganese, cobalt, palladium, platinum, tin, and mixtures thereof. Preferred metals include: zinc, copper, and mixtures thereof.

3. Oxidized Carbohydrate

The metal complex of an oxidized carbohydrate component of the compositions herein comprise oxidized carbohydrates. The oxidized carbohydrates may be used either in the dextro-rotary (D) or the levo-rotary (L) form. They may be substituted or un-substituted. When substituted, the oxidized carbohydrates useful herein may be amino-substituted, amido-substituted, phospho-substituted, or any mixture thereof. As described below, oxidized carbohydrates which are sulpho-substituted may optionally be included as additional ingredients.

The oxidized carbohydrates for use herein include substituted or un-substituted monosaccharides, disaccharides, oligosaccharides, polysaccharides, and mixtures thereof. Suitable oxidized carbohydrates for use herein include, but are not limited to: oxidized aldoses, oxidized ketoses, oxidized trioses, oxidized tetroses, oxidized pentoses, oxidized hexoses, and mixtures thereof.

Specific examples of oxidized monosaccharides for use herein include, but are not limited to: ribonic acid, ribulonic acid, arabinonic acid, xylonic acid, xylulonic acid, lyxonic acid, allonic acid, altronic acid, gluconic acid, mannonic acid, gulonic acid, idonic acid, galactonic acid, talonic acid, glucoheptonic acid, psiconic acid, fructonic acid, sorbonic acid, tagatonic acid, and mixtures thereof.

Specific examples of oxidized disaccharides for use herein include, but are not limited to: lactobionic acid, maltobionic acid, isomaltobionic acid, cellobionic acid, and mixtures thereof.

Specific examples of oxidized oligosaccharides for use herein include, but are not limited to: oxidized malto-oligosaccharide, oxidized cello-oligosaccharide, and mixtures thereof. Specific examples of oxidized polysaccharides for use herein include, but are not limited to: oxidized cellulose; chitin; gum arabic; gum karaya; gum xanthan; oxidized gum guar; oxidized locust bean gum; oxidized agars; oxidized algins; oxidized gellan gum; and mixtures thereof.

4. Preferred Metal Complexes of Oxidized Carbohydrates

Specific examples of preferred metal complexes of oxidized carbohydrates for use herein include, but are not limited to: lithium gluconate; copper gluconate; zinc galactonate; copper galactonate; zinc glucuronate; copper glucuronate; zinc galacturonate; copper galacturonate; zinc glucarate; copper glucarate; zinc galactarate; copper galactarate; zinc glucoheptonate; copper glucoheptonate; lithium lactobionate; sodium lactobionate; zinc lactobionate; copper lactobionate; lithium maltobionate; zinc maltobionate; copper maltobionate; lithium cellobionate; zinc cellobionate; copper cellobionate; and zinc alginate. Preferred are lithium lactobionate; sodium lactobionate; zinc lactobionate; copper lactobionate; lithium maltobionate; zinc maltobionate; copper maltobionate; lithium cellobionate; zinc cellobionate; and copper cellobionate. Highly preferred is zinc lactobionate. As described below, zinc gluconate and manganese gluconate are preferred metal complexes of oxidized carbohydrates for use as additional optional ingredients.

Lithium gluconate can be represented by the following structure:

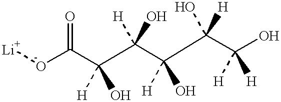

Zinc lactobionate can be represented by the following structure:

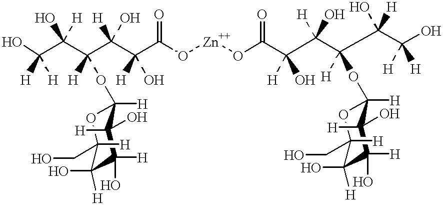

5. Solubility of Metal Complexes of Oxidized Carbohydrates

While not being limited by theory, it is believed that there may be a positively proportional relationship between the solubility in water of metal complexes of oxidized carbohydrates and their hair growth regulation efficacy, i.e. the greater the solubility in water, the greater the efficacy. Further, it is believed that metal complexes of oxidized polysaccharides have a greater solubility in water than metal complexes of oxidized disaccharides, which are believed to have a greater solubility in water than metal complexes of monosaccharides. Typically the metal complexes of oxidized carbohydrates of the present invention, will have a solubility, at ambient conditions, in water of at least about 5%, by weight, preferably at least about 10%, more preferably at least about 40%, more preferably at least about 50%, more preferably at least about 60%. As used herein, the “solubility” of a solute is a quantity that will dissolve in a given amount of solvent.

B. Vehicle

The method of the present invention utilizes compositions which comprise as an essential component, a vehicle for the metal complex of an oxidized carbohydrate, in an amount sufficient to carry an effective amount of at least one metal complex of an oxidized carbohydrate onto or into the body in an amount which is sufficiently effective to regulate hair growth. Typically such amount will range from about 0.1% to about 99.999%, by weight of the composition, preferably from about 25% to about 99.99%, more preferably from about 50% to about 99.9%, more preferably from about 75% to about 99%, more preferably from about 85% to about 99%.

The vehicle can comprise a solid, semi-solid or liquid cosmetically and/or physiologically acceptable vehicle, to enable the metal complex of an oxidized carbohydrate to be conveyed to the skin at an appropriate concentration. As used herein, “pharmaceutically-acceptable” means that drugs, medications or inert ingredients which the term describes are suitable for use in humans and lower animals without undue toxicity, incompatibility, instability, irritation, allergic response, and the like. As used herein, “cosmetically acceptable” means that ingredients which the term describes are suitable for use in contact with the skin or hair of humans and lower animals without undue toxicity, incompatibility, instability, irritation, allergic response and the like.

The nature of the vehicle will depend upon the method chosen for administration of the composition. The vehicle can itself be inert or it can possess cosmetic or pharmaceutical benefits of its own. When the compositions are to be applied topically, such vehicles will act as diluents, dispersants, or solvents for the metal complex of an oxidized carbohydrates, which therefore ensure that they can be applied to and distributed evenly over the hair and/or scalp at an appropriate concentration. The vehicle will preferably be one which can aid penetration of the metal complex of an oxidized carbohydrates into the skin to reach the immediate environment of the hair bulb. For all types of application, such vehicles should be physically and chemically compatible with the essential components described herein, and should not otherwise unduly impair product stability, aesthetics or performance. Vehicles suitable for use herein alone or in combination include: solvents; thickeners; propellants; powders; fillers; plasticizers; lubricants; and emollients and humectants.

1. Solvents

The vehicle of the compositions of the present invention can comprise alone or in combination with other vehicle ingredients, solvents. Generally, solvents suitable for use in the compositions herein are either water or are selected to be miscible with water and innocuous to the skin. Solvents suitable for use herein include, but are not limited to: water; C₁ to C₂₀ mono- or poly-hydric alcohols and their ethers, preferred are C₂ to C₃ mono- and di- hydric alcohols, particularly ethanol, isopropanol, n-propanol, and butanol; propylene glycol; ethylene glycol monoethyl ether; glycerine; methylene chloride; diethylene glycol monobutyl ether; diethylene glycol monoethyl ether; dimethyl sulphoxide; dimethyl formamide; tetrahydrofuran; propylene glycol; and mixtures thereof. Preferred solvents for use herein include: water, ethanol, isopropanol, propylene glycol, and mixtures thereof. When the solvent includes propylene glycol, it will typically contain it at a level of at least about 5%, by weight, preferably at least about 8%, more preferably at least about 10%, and typically the level will range from about 5% to about 20%, preferably from about 8% to about 15%, more preferably from about 10% to about 15%. It is believed that the presence of at least about 5% propylene glycol may improve penetration, and thereby, efficacy, of the metal-oxidized carbohydrate complex. When topically applied, propylene glycol's presence may also improve the appearance and/or the feel of the composition, on skin after drying.

2. Thickeners

The vehicle of the compositions of the present invention can comprise alone or in combination with other vehicle ingredients, thickening agents. Typically, such thickening agents when present, will be present at a level of from about 0.05% to about 20%, by weight of the composition, preferably from about 0.1% to about 10%, more preferably from about 0.5% to about 5%. It should be understood that under certain circumstances the thickening function may be accomplished by a material also serving as a silicone or emollient. For instance, silicone gums in excess of 10 centistokes and esters such as glycerol stearate impart this dual functionality. Thickening agents suitable for use in the compositions herein include, but are not limited to: oleic acid; cetyl alcohol; oleyl alcohol; sodium chloride; cetearyl alcohol; stearyl alcohol; synthetic thickeners such as those available under the tradenames ACULYN (RTM) and SALCARE (RTM) and ELFACOS (RTM), and those cross-linked polyacrylate materials available under the trademark Carbopol (RTM) from the B. F. Goodrich Company; and mixtures thereof. Some particular thickeners for use herein are ACULYN (RTM) 22 steareth-20 methacrylate copolymer, ACULYN (RTM) 33 anionic acrylic copolymer, ACULYN (RTM) 44 polyurethane resin, and ACULYN (RTM) 46 hydrophobically modified nonionic polyol and others, which are available from ISP (International Specialty Products). Also suitable are the SALCARE (RTM) series of thickeners (SC80, 81, 91, 92, 95, 96 AST) available from Ciba Specialty Chemicals. Also suitable are the series of thickeners available from Akzo Nobel such as ELFACOS (RTM) GT 282S ceteareth-60 myristyl glycol, ELFACOS (RTM) GT 282 L ceteareth-60 myristyl glycol, ELFACOS (RTM) T211 PPG 14 Laureth-60 Isophoryl dicarbamate, and ELFACOS (RTM) T212 PPG-14 Palmeth-60 Hexyl Dicarbamate. Additional thickening agents suitable for use herein include: sodium alginate; gum arabic; cellulose derivatives, such as ethylcellulose, methylcellulose, hydroxypropyl cellulose, hydroxypropylmethylcellulose, and carboxymethylcellulose, or the sodium salt of carboxymethylcellulose; acrylic polymers, such as carboxyvinyl polymer; acrylic resins, such as EUDRAGIT (RTM) RL30D, available from Rohm Pharma GmbH Weiderstadt, West Germany; polyvinylpyrrolidone or other commercially available film-coating preparations, such as DRI-KLEAR (RTM), available form Crompton & Knowles Corp., Mahwah, N.J., USA, or OPADRY (RTM), available from Colorcon, West Point, Pa., USA. Also suitable for use herein as thickening agents are: gums, such as xanthan gum, guar gum, locust bean gum; carrageenan; gelatin; karaya; pectin; Biopolymer PS 87; clays, such as hectorites and bentonites; and mixtures thereof.

Additional thickeners suitable for use herein are those disclosed in: WO 99/37,047 (nonionic polyurethanes and/or cationic polymers); EP 0,875,237A2 (hydrophobically modified nonionic polyols and polyethoxylated urethane); WO 99/36,047 (polyurethane polymers and/or cationic conditioning agents); WO 98/03,150 (nonionic amphophilic polymers having at least one fatty chain); and U.S. Pat. No. 5,281,654 (mixture of polyurethanes), all of which descriptions are incorporated herein by reference.

3. Propellants

The vehicle of the compositions of the present invention can comprise alone or in combination with other vehicle ingredients, propellants. Propellants suitable for use herein include, but are not limited to: propane; butane; isobutane; dimethyl ether; carbon dioxide; nitrogen; nitrous oxide; and mixtures thereof.

4. Powders

The vehicle of the compositions of the present invention can comprise alone or in combination with other vehicle ingredients, powders. Powders suitable for use herein include, but are not limited to: chalk; talc; fullers earth; kaolin; starch; gums; colloidal silicon dioxide; sodium polyacrylate; tetra alkyl and/or trialkyl aryl ammonium smectites; chemically modified magnesium aluminum silicate; organically modified montmorillonite clay; hydrated aluminum silicate; fumed silica; TiO₂ and TiO₂-coated mica, and mixtures thereof.

5. Fillers

The vehicle of the compositions of the present invention can comprise alone or in combination with other vehicle ingredients, fillers. Fillers suitable for use herein include, but are not limited to: lactose, sucrose, maltodextrin, mannitol, starch, dicalcium phosphate and microcrystalline cellulose.

6. Plasticizers

The vehicle of the compositions of the present invention can comprise alone or in combination with other vehicle ingredients, plasticizers. Plasticizers suitable for use herein include, but are not limited to: polyethylene glycol; propylene glycol; dibutyl phthalate; castor oil; acetylated monoglycerides; triacetin; and mixtures thereof.

7. Lubricants

The vehicle of the compositions of the present invention can comprise alone or in combination with other vehicle ingredients, lubricants. Lubricants suitable for use herein include, but are not limited to: magnesium stearate; stearic acid; talc; and mixtures thereof.

8. Emollients and Humectants

The vehicle of the compositions of the present invention can comprise alone or in combination with other vehicle ingredients, emollients and humectants. Some emollients and humectants which are useful as being all or part of the vehicle herein include, but are not limited to: esters; fatty alcohols and acids; polyols; hydrocarbons; non-volatile silicones; waxes; animal fats; vegetable oils; and mixtures thereof.

One class of emollients and humectants suitable for use herein are esters, such class includes: C1-C30 alcohol esters of C1-C30 carboxylic acids and of C2-C30 dicarboxylic acids, including straight and branched chain materials as well as aromatic derivatives can also be used herein. Also useful herein are esters such as monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, and propylene glycol diesters of C1-C30 carboxylic acids. Straight chain, branched chain and aryl carboxylic acids are included herein. Also useful are propoxylated and ethoxylated derivatives of these materials.

Non-limiting examples of esters useful herein include, but are not limited to: diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprylic/capric triglyceride, PEG-6 caprylic/capric triglyceride, PEG-8 caprylic/capric triglyceride, and mixtures thereof.

Also useful herein are various C1-C30 monoesters and polyesters of sugars and related materials. These esters are derived from a sugar or polyol moiety and one or more carboxylic acid moieties. Depending on the constituent acid and sugar, these esters can be in either liquid or solid form at room temperature. Examples of liquid esters include, but are not limited to: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include, but are not limited to: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C18 mono- and/or di-unsaturated and behenic, in a molar ratio of unsaturates:behenic of 1:7 to 3:5. A particularly preferred solid sugar polyester is the octaester of sucrose in which there are about 7 behenic fatty acid moieties and about 1 oleic acid moiety in the molecule. Other materials include cottonseed oil or soybean oil fatty acid esters of sucrose. The ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Patent No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985; all of which are incorporated by reference herein in their entirety.

Suitable fatty alcohols and acids for use herein include, but are not limited to those compounds having from 10 to 20 carbon atoms. Preferred are such compounds as cetyl, myristyl, palmitic and stearyl alcohols and acids.

Among the polyols which may comprise all or part of the vehicle herein are linear and branched chain alkyl polyhydroxyl compounds. Preferred polyols include propylene glycol, sugars having up to about 12 carbons atoms, sugar alcohols having up to about 12 carbon atoms, and mixtures thereof, glycerin, polypropylene glycols, polyethylene glycols, ethyl hexane diol, hexylene glycols, and mixtures thereof.

Specific examples of polyols useful herein include, but are not limited to: materials such as sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, polypropylene glycols such as PPG-9, PPG-12, PPG-15, PPG-17, PPG-20, PPG-26, PPG-30, PPG-34; alkoxylated glucose; hyaluronic acid; and mixtures thereof.

Also useful are materials such as: urea; guanidine; glycolic acid and glycolate salts (e.g. ammonium and quaternary alkyl ammonium); lactic acid and lactate salts (e.g. ammonium and quaternary alkyl ammonium); cholesterol; and mixtures thereof.

Also useful are materials such as aloe vera in any of its variety of forms (e.g., aloe vera gel), chitin, starch-grafted sodium polyacrylates such as SANWET (RTM) IM-1000, IM-1500, and IM-2500 (available from Celanese Superabsorbent Materials, Portsmouth, Va., USA); lactamide monoethanolamine; acetamide monoethanolamnine; and mixtures thereof. Also useful are propoxylated glycerols described in U.S. Pat. No. 4,976,953, to Orr et al., issued Dec. 11, 1990, which is incorporated by reference herein in its entirety.

Suitable hydrocarbons for use in the vehicle herein are straight and branched chain hydrocarbons having from 7 to 40 carbon atoms. Non-limiting examples include mineral oil, petrolatum, squalene, isoparaffins. dodecane, isododecane, hydrogenated polyisobutylene, docosane (i.e. a C₂₂ hydrocarbon), hexadecane, isohexadecane (a commercially available hydrocarbon sold as PERMETHYL (RTM) 101A by Presperse, South Plainfield, N.J., USA).

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p.415417 (1993), which are incorporated by reference herein in their entirety.

Petrolatum, which is also known as petroleum jelly, is a colloidal system of non-straight-chain solid hydrocarbons and high-boiling liquid hydrocarbons, in which most of the liquid hydrocarbons are held inside the micelles. See The Merck Index, Tenth Edition, Entry 7047, p. 1033 (1983); Schindler, Drug. Cosmet. Ind., vol. 89, 36-37, 76, 78-80, 82 (1961); and International Cosmetic Ingredient Dictionary, Fifth Edition, vol. 1, p. 537 (1993), which are incorporated by reference herein in their entirety.

Non-volatile silicones such as polydialkylsiloxanes, polydiarylsiloxanes, and polyalkarylsiloxanes are also useful herein. These silicones are disclosed in U.S. Pat. No. 5,069,897, to Orr, issued Dec. 3, 1991, which is incorporated by reference herein in its entirety. The polyalkylsiloxanes correspond to the general chemical formula R₃SiO[R₂SiO]ₓSiR₃ wherein R is an alkyl group (preferably R is methyl or ethyl, more preferably methyl) and x is an integer up to about 500, chosen to achieve the desired molecular weight. Commercially available polyalkylsiloxanes include the polydimethylsiloxanes, which are also known as dimethicones, non-limiting examples of which include the VISCASIL (RTM) series sold by General Electric Company and the DOW CORNING (RTM) 200 series sold by Dow Corning Corporation. Specific examples of polydimethylsiloxanes useful herein include DOW CORNING (RTM) 225 fluid having a viscosity of 10 centistokes and a boiling point greater than 200° C., and DOW CORNING (RTM) 200 fluids having viscosities of 50, 350, and 12,500 centistokes, respectively, and boiling points greater than 200° C. Also useful are materials such as trimethylsiloxysilicate, which is a polymeric material corresponding to the general chemical formula [(CH₂)₃SiO_{0.5}]ₓ[SiO₂]_{y}, wherein x is an integer from about 1 to about 500 and y is an integer from about 1 to about 500. A commercially available trimethylsiloxysilicate is sold as a mixture with dimethicone as DOW CORNING (RTM) 593 fluid. Also useful herein are dimethiconols, which are hydroxy terminated dimethyl silicones. These materials can be represented by the general chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer up to about 500, chosen to achieve the desired molecular weight. Commercially available dimethiconols are typically sold as mixtures with dimethicone or cyclomethicone (e.g. DOW CORNING (RTM) 1401, 1402, and 1403 fluids). Also useful herein are polyalkylaryl siloxanes, with polymethylphenyl siloxanes having viscosities from about 15 to about 65 centistokes at 25° C. being preferred. These materials are available, for example, as SF 1075 methylphenyl fluid (sold by General Electric Company) and 556 Cosmetic Grade phenyl trimethicone fluid (sold by Dow Corning Corporation).

Waxes which are useful in being all or part of the vehicle in the compositions herein include those set forth in CTFA Cosmetic Ingredient Handbook, Second Edition, 1992, pp. 535, which is herein incorporated by reference. Specific examples include beeswax, carnauba, candelilla wax, jojoba wax, lanolin wax, ozokerite, paraffin wax, and mixtures thereof.

Animal fats, vegetable oils and hydrogenated vegetable oils, and vegetable oil adducts are also potentially useful herein as all or part of the vehicle.

Examples of vegetable oils and hydrogenated vegetable oils include, but are not limited to: safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.

Some preferred emollients and humectants for use herein include, but are not limited to: glycerine; sorbitol; sodium 2-pyrrolidone-5-carboxylate; soluble collagen; dibutyl phthalate; gelatin; stearyl alcohol; glyceryl monoricinoleate; glyceryl monostearate; propane-1,2-diol; butane-1,3-diol; mink oil; cetyl alcohol; isopropyl isostearate; stearic acid; isobutyl palmitate; isocetyl stearate; oleyl alcohol; isopropyl laurate; hexyl laurate; decyl oleate; octadecan-2-ol; isocetyl alcohol; cetyl palmitate; dimethylpolysiloxane; di-n-butyl sebacate; isopropyl myristate; isopropyl palmitate; isopropyl stearate; butyl stearate; polyethylene glycol; triethylene glycol; lanolin; sesame oil; coconut oil; arachis oil; castor oil; acetylated lanolin alcohols; petroleum; mineral oil; butyl myristate; isostearic acid; palmitic acid; isopropyl linoleate; lauryl lactate; myristyl lactate; decyl oleate; myristyl myristate; and mixtures thereof.

### II. Optional Components

The compositions of the present invention may, in some embodiments, further comprise additional optional components known or otherwise effective for use in topically applied personal care products. Surfactants, conditioning agents, cationic polymers, anti-dandruff actives, activity enhancers, penetration enhancers, non-oxidative and other dyes, suspending agents, non-steroidal anti-inflammatory drugs, topical anesthetics, sunscreen actives, flavoring agents, preservatives, sweeteners, and other optional components are described in detail below. Any optional component(s) should be physically and chemically compatible with the essential components of the composition, and should not otherwise unduly impair product stability, aesthetics or performance. Zinc gluconate and manganese gluconate are preferred metal complexes of oxidized carbohydrates for use as additional optional ingredients.

A. Surfactant

The compositions utilized in the method of the present invention, may, in some embodiments contain a surfactant suitable for application to the hair or skin, particularly when the compositions are to be applied topically, although surfactant may be used in any other form, adjusting the amount of surfactant present according to the desired effect for that particular form. Typically, such concentrations will range from about 5% to about 50%, by weight of the composition, preferably from about 8% to about 30%, more preferably from about 10% to about 25%, most preferably from about 12% to about 18%. When present, the surfactant is believed to provide cleaning and lather performance to the composition. Additionally, when an anionic detersive surfactant is used in combination with a cationic polymer, a coacervate is formed upon aqueous dilution, which is believed to be important in providing efficacy benefits. Such surfactants should be physically and chemically compatible with the essential components described herein, and should not otherwise unduly impair product stability, aesthetics or performance. Examples of surfactants which may be suitably employed in the compositions herein include: anionic, non-ionic, amphoteric or zwitterionic, cationic, and mixtures thereof.

1. Anionic surfactants

The anionic surfactant component, when present, can comprise an anionic detersive surfactant, a zwitterionic or an amphoteric detersive surfactant having an attached moiety that is anionic at the pH of the composition, or a combination thereof; preferably an anionic surfactant. Examples of anionic detersive surfactants which may be suitably employed in the compositions herein include, but are not limited to: sulfates, sulfonates, sarcosinates and sarcosine derivatives.

a. Sulfates

Suitable anionic surfactants for use in the compositions of the present invention are the alkyl and alkyl ether sulfates. These surfactants have the respective formulae ROSO₃M and R(C₂H₄O)ₓOSO₃M, wherein R is alkyl or alkenyl from about C₈ to about C₁₈, x is an integer having a value from 1 to 10, and M is a cation selected from the group consisting of electropositive covalently bonded moieties (e.g. ammonium), alkanolamines (e.g. triethanolamine), monovalent metals (e.g. sodium or potassium), polyvalent metal cations (e.g. magnesium and calcium) and mixtures thereof. The cation M should be selected such that the anionic detersive surfactant component is water soluble. Solubility of the surfactant will depend upon the particular anionic detersive surfactants and cations chosen.

Preferably, R is from about C₈ to about C₁₈, more preferably from about C₁₀ to about C₁₆, most preferably from about C₁₂ to about C₁₄, in both the alkyl and alkyl ether sulfates. The alkyl ether sulfates are typically made as condensation products of ethylene oxide and monohydric alcohols from about C₈ to about C₂₄. The alcohols can be synthetic or they can be derived from fats, e.g., coconut oil, palm kernel oil, and tallow. Lauryl alcohol and straight chain alcohols derived from coconut oil or palm kernel oil are preferred. Such alcohols are reacted with from 0 to about 10, preferably from about 2 to about 5, most preferably about 3, moles of ethylene oxide. The resulting mixture of molecular species will have, for example, an average of 3 moles of ethylene oxide per mole of alcohol, and is sulfated and neutralized.

Non-limiting examples of alkyl ether sulfates which may be used in the shampoo compositions of the present invention include sodium and ammonium salts of coconut alkyl triethylene glycol ether sulfate, tallow alkyl triethylene glycol ether sulfate, and tallow alkyl hexaoxyethylene sulfate. Preferred alkyl ether sulfates are those comprising a mixture of individual compounds, wherein the compounds in the mixture have an average alkyl chain length from about C₁₀ to about C₁₆ and an average degree of ethoxylation of from about 1 to about 4 moles of ethylene oxide.

Specific examples of preferred alkyl sulfates include, but are not limited to, ammonium lauryl sulfate, ammonium cocoyl sulfate, potassium lauryl sulfate, potassium cocoyl sulfate, sodium lauryl sulfate, sodium cocoyl sulfate, monoethanolamine lauryl sulfate, monoethanolamine cocoyl sulfate, diethanolamine lauryl sulfate, triethanolamine lauryl sulfate, triethylamine lauryl sulfate, sodium cetyl sulfate, ammonium cetyl sulfate, and mixtures thereof. Especially preferred is ammonium lauryl sulfate.

Specific examples of preferred alkyl ether sulfates include, but are not limited to, ammonium laureth sulfate, potassium laureth sulfate, sodium laureth sulfate, monoethanolamine laureth sulfate, diethanolamine laureth sulfate, triethanolamine laureth sulfate, triethylamine laureth sulfate, sodium trideceth sulfate, and mixtures thereof. Especially preferred is ammonium laureth sulfate.

Still another class of sulfate surfactants suitable for use in the for use in the hair growth regulatings of the present invention are the sulfated glycerides, an example of which includes, but is not limited to, lauric monoglyceride sodium sulfate.

b. Sulfonates

Also suitable for use in the hair growth regulating compositions of the present invention are those anionic detersive surfactants known as olefin sulfonates. As used herein, the term “olefin sulfonates” refers to compounds which can be produced by the sulfonation of α-olefins by means of uncomplexed sulfur trioxide, followed by neutralization of the acid reaction mixture in conditions such that any sultones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, and the like, when used in the liquid form, or by air, nitrogen, gaseous SO₂, and the like, when used in the gaseous form. The α-olefms from which the olefin sulfonates are derived are mono-olefins which are from about C₁₀ to about C₂₄, preferably from about C₁₂ to about C₁₆. Preferably, they are straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefms and impurities in the olefin stock and side reactions during the sulfonation process. A non-limiting example of such an α-olefin sulfonate mixture is described in U.S. Pat. No. 3,332,880, which description is incorporated herein by reference. Another class of sulfonates suitable for use in the hair growth regulating compositions of the present invention is those anionic detersive surfactants known as β-alkyloxy alkane sulfonates. These surfactants conform to the general Formula (III):

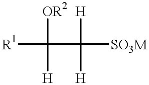

where R¹ is a straight chain alkyl group from about C₆ to about C₂₀, R² is a lower alkyl group from about C₁ to about C₃, preferably C₁, and M is a water-soluble cation, as described above.

Still other sulfonates suitable for use in the hair growth regulating compositions of the present invention are those anionic detersive surfactants known as alkyl aryl sulfonates. Non-limiting examples of alkyl aryl sulfonates include sodium tridecyl benzene sulfonate, sodium dodecyl benzene sulfonate, and mixtures thereof.

Other suitable sulfonates for use in the hair growth regulating compositions of the present invention are the water-soluble salts of organic, sulfuric acid reaction products conforming to the formula [R¹—SO₃—M ] where R¹ is a straight or branched chain, saturated, aliphatic hydrocarbon radical from about C₈ to about C₂₄, preferably about C₁₀ to about C₁₈; and M is a cation described above. Non-limiting examples of such anionic detersive surfactants are the salts of an organic sulfuric acid reaction product of a hydrocarbon of the methane series, including iso-, neo-, and n-paraffins, and a sulfonating agent, e.g., SO₃, H₂SO₄, obtained according to known sulfonation methods. The sulfonation methods may include bleaching and hydrolysis. The salts are preferably from about C₈ to about C24; more preferably from about C₁₂ to about C₁₈. Preferred are alkali metal and ammonium sulfonated C₁₀ to C₁₈ n-paraffins.

Still other suitable sulfonates for use in the hair growth regulating compositions of the present invention are the reaction products of fatty acids, which are esterified with isethionic acid, and then neutralized with sodium hydroxide. Preferred fatty acids are those derived from coconut oil or palm kernel oil. Also suitable are the sodium or potassium salts of fatty acid amides of methyl tauride in which the fatty acids are derived from coconut oil or palm kernel oil. Other similar anionic surfactants are described in U.S. Pat. No. 2,486,921; U.S. Pat. No. 2,486,922; and U.S. Pat. No. 2,396,278, which descriptions are incorporated herein by reference.

Other sulfonates suitable for use in the hair growth regulating compositions of the present invention are the succinnates, examples of which include, but are not limited to, disodium N-octadecylsulfosuccinate, disodium lauryl sulfosuccinate, diammonium lauryl sulfosuccinate, tetrasodium N-(1,2-di-carboxyethyl)-N-octadecylsulfosuccinnate, diamyl ester of sodium sulfosuccinic acid, dihexyl ester of sodium sulfosuccinic acid, dioctyl esters of sodium sulfosuccinic acid, and mixtures thereof.

C. Sarcosinates and Sarcosine Derivatives

Also suitable for use in the hair growth regulating compositions of the present invention are those anionic detersive surfactants known as sarcosinates and sarcosine derivatives. Sarcosinates are the derivatives of sarcosine (also known as N-methyl glycine), acylated with a fatty acid chloride. They conform to the general Formula (IV):

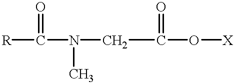

wherein RCO— is a fatty acid radical, R being R an alkyl or alkenyl having from about 10 to about 20 carbon atoms, and wherein X is either hydrogen (acid form) or a cationic species, such as Na⁺or TEA⁺(salt form), or a water-soluble cation such as ammonium, sodium, potassium or trialkanolamine. Non-limiting examples of sarcosinates and sarcosine derivatives include: sodium lauryl sarcosinate, lauryl sarcosine, cocoyl sarcosine, sodium lauroyl sarcosinate, and mixtures thereof. A preferred sarcosinate is sodium lauryl sarcosinate.

d. Alkoyl Isethionates

Another class of anionic surfactants which may be useful in the compositions herein are the alkoyl isethionates, which typically conform to the formula RCO—OCH₂CH₂SO₃M, wherein R is alkyl or alkenyl having from about 10 to about 30 carbon atoms, and M is a water-soluble cation such as ammonium, sodium, potassium and triethanolamine. Non-limiting examples of these isethionates include, but are not limited to: ammonium cocoyl isethionate, sodium cocoyl isethionate, sodium lauroyl isethionate, and mixtures thereof.

e. Soaps of Fatty Acids

Other anionic materials useful herein are soaps (i.e. alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853, which description is incorporated herein by reference.

f. Taurates

Also useful are taurates which are based on taurine, which is also known as 2-aminoethanesulfonic acid. Examples of taurates include N-alkyltaurines such as the one prepared by reacting dodecylamine with sodium isethionate according to the teaching of U.S. Patent 2,658,072 which is incorporated herein by reference in its entirety.

Also suitable for use in the compositions of the present invention are: phosphates, such as monoalkyl, dialkyl, and trialkylphosphate salts; alkyl glyceryl ether sulfonates; methyl acyl taurates; fatty acyl glycinates; N-acyl glutamates; alkyl ethoxysulphosuccinates; alpha-sulfonated fatty acids, their salts and/or their esters; alkyl ethoxy carboxylates; alkyl phosphate esters; ethoxylated alkyl phosphate esters; alkyl sulphates; acyl sarcosinates; fatty acid/protein condensates; and mixtures thereof. 2. Nonionic Surfactants

The compositions of the invention may also comprise water-soluble nonionic surfactant(s). Surfactants of this class include C₁₂ to C₁₄ fatty acid mono-and di- ethanolamides, sucrose polyester surfactants and polyhydroxy fatty acid amide surfactants having the general formula (V):

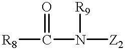

The preferred N-alkyl, N-alkoxy or N-aryloxy, polyhydroxy fatty acid amide surfactants according to the above formula are those in which R8 is C₅ to C₃₁ hydrocarbyl, preferably C₆ to C₁₉ hydrocarbyl, including straight-chain and branched chain alkyl and alkenyl, or mixtures thereof and R₉ is typically hydrogen, C₁ to C₈ alkyl or hydroxyalkyl, preferably methyl, or a group of formula —R¹—O—R² (wherein R¹ is C₂ to C₈ hydrocarbyl including straight-chain, branched-chain and cyclic (including aryl), and is preferably C₂ to C₄ alkylene, R² is C₁ to C₈ straight-chain, branched-chain and cyclic hydrocarbyl including aryl and oxyhydrocarbyl, and is preferably C₁ to C₄ alkyl, especially methyl, or phenyl)). Z₂ is a polyhydroxyhydrocarbyl moiety having a linear hydrocarbyl chain with at least 2 (in the case of glyceraldehyde) or at least 3 hydroxyls (in the case of other reducing sugars) directly connected to the chain, or an alkoxylated derivative (preferably ethoxylated or propoxylated) thereof. Z₂ preferably will be derived from a reducing sugar in a reductive amination reaction, more preferably Z₂ is a glycityl moiety. Suitable reducing sugars include glucose, fructose, maltose, lactose, galactose, mannose, and xylose, as well as glyceraldehyde. A preferred surfactant corresponding to the above structure is coconut alkyl N-methyl glucoside amide (i.e., wherein the R₈CO- moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety. As raw materials, high dextrose corn syrup, high fructose corn syrup, and high maltose corn syrup can be utilized as well as the individual sugars listed above. These corn syrups may yield a mix of sugar components for Z₂. It should be understood that it is by no means intended to exclude other suitable raw materials. Z₂ preferably will be selected from the group consisting of —CH₂—(CHOH)ₙ—CH₂OH, —CH(CH₂OH)—(CHOH)ₙ₋₁—CH₂H, CH₂(CHOH)₂(CHOR′)—CHOH)—CH₂OH, wherein n is an integer from 1 to 5, inclusive, and R′ is H or a cyclic mono- or polysaccharide, and alkoxylated derivatives thereof. As noted, most preferred are glycityls wherein n is 4, particularly —CH₂—(CHOH)₄—CH₂OH.

A highly preferred polyhydroxy fatty acid amide has the formula R₈(CO)N—(CH₃)CH₂(CHOH)₄CH₂OH wherein R₈ is a C₆ to C₁₉ straight chain alkyl or alkenyl group. In compounds of the above formula, R₈—CO—N— can be, for example, cocoamide, stearamide, oleamide, lauramide, myristamide, capricamide, palmiarnide, tallowamide, and the like.

Suitable oil derived nonionic surfactants for use herein include water soluble vegetable and animal-derived emollients such as triglycerides with a polyethylene glycol chain inserted; ethoxylated mono- and di- glycerides, polyethoxylated lanolins and ethoxylated butter derivatives. One preferred class of oil-derived nonionic surfactants for use herein have the general formula below (VI):

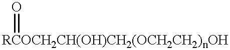

wherein n is from about 5 to about 200, preferably from about 20 to about 100, more preferably from about 30 to about 85, and wherein R comprises an aliphatic radical having on average from about 5 to 20 carbon atoms, preferably from about 7 to 18 carbon atoms.

Suitable ethoxylated oils and fats of this class include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, preferably glyceryl tallowate and glyceryl cocoate.

Preferred for use herein are polyethylene glycol based polyethoxylated C₉-C₁₅ fatty alcohol nonionic surfactants containing an average of from about 5 to about 50 ethyleneoxy moieties per mole of surfactant.

Suitable polyethylene glycol based polyethoxylated C₉-C₁₅ fatty alcohols suitable for use herein include C₉-C₁₁ Pareth-3, C₉-C₁₁ Pareth-4, C₉-C₁₁ Pareth-5, C₉-C₁₁ Pareth-6, C₉-C₁₁ Pareth-7, C₉-C₁₁ Pareth-8, C₁₁-C₁₅ Pareth-3, C₁₁-C₁₅ Pareth-4, C₁₁-C₁₅ Pareth-5, C₁₁-C₁₅ Pareth-6, C₁₁-C₁₅ Pareth-7, C₁₁-C₁₅ Pareth-8, C₁₁-C₁₅ Pareth-9, C₁₁-C₁₅ Pareth-10, C₁₁-C₁₅ Pareth-11, C₁₁-C₁₅ Pareth-12, C₁₁-C₁₅ Pareth-13 and C₁₁-C₁₅ Pareth-14. PEG 40 hydrogenated castor oil is commercially available under the tradename CREMOPHOR (RTM) from BASF. PEG 7 glyceryl cocoate and PEG 20 glyceryl laurate are commercially available from Henkel under the tradenames CETIOL (RTM) HE and LAMACIT (RTM) GML 20, respectively. C9-C₁₁Pareth-8 is commercially available from Shell, Ltd., under the tradename DOBANOL (RTM) 91-8. Particularly preferred for use herein are polyethylene glycol ethers of ceteryl alcohol such as Ceteareth 25 which is available from BASF under the trade name CREMAPHOR (RTM) A25.

Also suitable for use herein are nonionic surfactants derived from composite vegetable fats extracted from the fruit of the Shea Tree (Butyrospermum Karkii Kotschy) and derivatives thereof. Similarly, ethoxylated derivatives of mango, cocoa and illipe butter may be used in compositions according to the invention. Although these are generally classified as ethoxylated nonionic surfactants, it is understood that a certain proportion may remain as non-ethoxylated vegetable oil or fat.

Other suitable oil-derived nonionic surfactants include ethoxylated derivatives of almond oil, peanut oil, rice bran oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil.

Also suitable for use herein are nonionic surfactants which are produced by the condensation of alkylene oxide groups (hydrophilic in nature) with an organic hydrophobic compound, which may be aliphatic or alkyl aromatic in nature, and which include alkyl glucosides and alkyl polyglucosides, which are defined as condensation products of long chain alcohols, e.g. C8-30 alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of such surfactants include decyl polyglucoside (available as APG 325 CS from Henkel Corporation) and lauryl polyglucoside (available as APG 600CS and 625 CS from Henkel Corporation).

Other examples of nonionic surfactants useful herein include amine oxides. Amine oxides correspond to the general formula R₁R₂R₃N→O, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

3. Amphoteric and Zwitterionic Surfactants

The term “amphoteric surfactant,” as used herein, is also intended to encompass zwitterionic surfactants, which are well known to formulators skilled in the art as a subset of amphoteric surfactants.

A wide variety of amphoteric surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Non-limiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as LONZAINE (RTM) 16SP from Lonza Corp.), lauryl bis-(2-hydroxyethyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines (wherein the RCONH(CH₂)₃ radical is attached to the nitrogen atom of the betaine), oleyl betaine (available as amphoteric VELVETEX (RTM) OLB-50 from Henkel), and cocamidopropyl betaine (available as VELVETEX (RTM) BK-35 and BA-35 from Henkel).

Examples of sultaines and hydroxysultaines include materials such as cocamidopropyl hydroxysultaine (available as MIRATAINE (RTM) CBS from Rhone-Poulenc).

Preferred for use herein are amphoteric surfactants having the following structure:

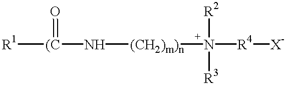

wherein R¹ is unsubstituted, saturated or unsaturated, straight or branched chain alkyl having from about 9 to about 22 carbon atoms. Preferably, R¹ has from about 11 to about 18 carbon atoms; more preferably from about 12 to about 18 carbon atoms; more preferably still from about 14 to about 18 carbon atoms; m is an integer from 1 to about 3, more preferably from about 2 to about 3, and more preferably about 3; n is either 0 or 1, preferably 1; R² and R³ are independently selected from the group consisting of alkyl having from 1 to about 3 carbon atoms, unsubstituted or mono-substituted with hydroxy, preferred R² and R³ are CH₃; X is selected from the group consisting of CO₂, SO₃ and SO₄; R⁴ is selected from the group consisting of saturated or unsaturated, straight or branched chain alkyl, unsubstituted or monosubstituted with hydroxy, having from 1 to about 5 carbon atoms. When X is CO₂, R⁴ preferably has 1 or 3 carbon atoms, more preferably 1 carbon atom. When X is SO₃ or SO₄, R⁴ preferably has from about 2 to about 4 carbon atoms, more preferably 3 carbon atoms.

Examples of amphoteric surfactants of the present invention include the following compounds:

Cetyl dimethyl betaine (this material also has the CTFA designation cetyl betaine)

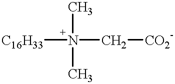

Cocamidopropylbetaine

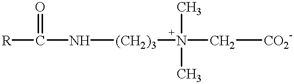

wherein R has from about 9 to about 13 carbon atoms

Cocamidopropyl hydroxy sultaine

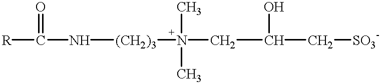

wherein R has from about 9 to about 13 carbon atoms,

Examples of other useful amphoteric surfactants are alkyliminoacetates, and iminodialkanoates and aminoalkanoates of the formulas RN[(CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-higher alkyl aspartic acids such as those produced according to the teaching of U.S. Pat. No. 2,438,091 which is incorporated herein by reference in its entirety; and the products sold under the name MIRANOL (RTM) and described in U.S. Pat. No. 2,528,378, which is incorporated herein by reference in its entirety. Other examples of useful amphoterics include amphoteric phosphates, such as coamidopropyl PG-dimonium chloride phosphate (commercially available as MONAQUAT (RTM) PTC, from Mona Corp.). Also useful are amphoacetates such as disodium lauroamphodiacetate, sodium lauroamphoacetate, and mixtures thereof.

4. Cationic Surfactants

The compositions of the invention may also comprise water-soluble cationic surfactant(s). Such surfactants are described in Surfactant Encyclopedia, Martin Reiger, (published by Cosmetics and Toiletries, ISBN 0-931710-294), which description is incorporated herein by reference. Non-limiting examples of cationic surfactants useful herein are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Cationic detersive surfactants suitable for use herein include, but are not limited to, surfactants containing quaternary nitrogen moieties. Examples of suitable cationic surfactants are those corresponding to the general Formula (VII):

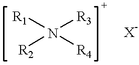

wherein R₁, R₂, R₃, and R4 are independently selected from a C₁ to C₂₂ aliphatic group or an aromatic, alkoxy, polyoxyalkylene, alkylamido, hydroxyalkyl, aryl or alkylaryl group having up to about 22 carbon atoms, preferably C₁ to C₂₂ alkyl; and X is a salt-forming anion, such as those selected from halogen (e.g. chloride, bromide), acetate, citrate, lactate, glycolate, phosphate nitrate, sulfate, and alkylsulfate radicals. The aliphatic groups can contain, in addition to carbon and hydrogen atoms, ether linkages, and other groups, such as amino groups. The longer chain (e.g. C₁₂ and higher) aliphatic groups can be saturated or unsaturated.

Preferred cationic detersive surfactants are those containing two long alkyl chains and two short alkyl chains or those containing one long alkyl chain and three short alkyl chains. Such long alkyl chains are preferably from C₁₂ to C₂₂, more preferably from C₁₆ to C₂₂. Such short alkyl chains are preferably from C₁ to C₃, more preferably from C₁ to C₂.

Alternatively, other useful cationic surfactants include amino-amides, wherein in the above structure R₁ is alternatively R₅CO—(CH₂)ₙ—, wherein R₅ is an alkyl group having from about 12 to about 22 carbon atoms, and n is an integer from about 2 to about 6, more preferably from about 2 to about 4, and most preferably from about 2 to about 3. Non-limiting examples of these cationic emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Non-limiting examples of quaternary ammonium salt cationic surfactants include those selected from the group consisting of cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl dimethyl ammonium chloride, distearyl methyl ammonium bromide, and mixtures thereof. Additional quaternary ammonium salts include those wherein the C12 to C22 alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C16 to C18 range. The term “coconut” refers to an alkyl group derived from a coconut fatty acid, which generally have mixtures of alkyl chains in the C12 to C14 range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

Preferred cationic surfactants useful herein include those selected from the group consisting of dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, and mixtures thereof.

B. Conditioning Agent

The hair growth regulating compositions of the present invention may comprise from about 0.01% to about 30%, by weight of the composition, preferably from about 0.1% to about 20%, more preferably from about 0. 1% to about 10%, most preferably from about 0.2% to about 6%, of a conditioning agent suitable for application to the hair or skin. It is believed that the conditioning agent provides improved conditioning benefits to the hair, particularly clean hair feel and wet rinse feel.

Suitable conditioning agents for use in the compositions herein are those conditioning agents characterized generally as silicones (e.g. silicone oils, cationic silicones, silicone gums, high refractive silicones, and silicone resins), organic conditioning oils (e.g. hydrocarbon oils, polyolefins, and fatty esters) or combinations thereof, or those conditioning agents which otherwise form liquid, dispersed, particles in the aqueous surfactant matrix herein. Such conditioning agents should be physically and chemically compatible with the essential components of the composition, and should not otherwise unduly impair product stability, aesthetics or performance.

The concentration of the conditioning agent in the hair bleaching composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

1. Silicones

The conditioning agent of the hair growth regulating compositions of the present invention is preferably an insoluble silicone conditioning agent. The silicone conditioning agent particles may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair (especially when high refractive index (e.g. above about 1.46) silicone conditioning agents are used (e.g. highly phenylated silicones).

The concentration of the silicone conditioning agent typically ranges from about 0.01% to about 10%, by weight of the composition, preferably from about 0.1% to about 8%, more preferably from about 0.1% to about 5%, most preferably from about 0.2% to about 3%. Non-limiting examples of suitable silicone conditioning agents, and optional suspending agents for the silicone, are described by the Procter & Gamble Company in U.S. Reissue Pat. No. RE 34,584, U.S. Pat. No. 5,104,646, and U.S. Pat. No. 5,106,609, all which are incorporated herein by reference in their entirety. The silicone conditioning agents for use herein preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, most preferably from about 100,000 to about 1,500,000 csk.

The dispersed, silicone conditioning agent particles typically have a number average particle diameter ranging from about 0.01 μm to about 50 μm. For small particle application to hair, the number average particle diameters typically range from about 0.01 μm to about 4 μm, preferably from about 0.01 μm to about 2 μm, more preferably from about 0.01 μm to about 0.5 μm. For larger particle application to hair, the number average particle diameters typically range from about 4 μm to about 50 μm, preferably from about 6 μm to about 30 μm, more preferably from about 9 μm to about 20 μm, most preferably from about 12 μm to about 18 μm. Conditioning agents having an average particle size of less than about 5 μm may deposit more efficiently on the hair.

Background material on silicones including sections discussing silicone fluids, gums, and resins, as well as manufacture of silicones, are found in Encyclopedia of Polymer Science and Engineering, vol. 15, 2d ed., pages 204-308, John Wiley & Sons, Inc. (1989), which description is incorporated herein by reference.

a. Silicone Oils

Silicone fluids include silicone oils, which are flowable silicone materials having a viscosity, as measured at 25° C., less than 1,000,000 csk, preferably from about 5 csk to about 1,000,000 csk, more preferably from about 10 csk to about 100,000 csk. Suitable silicone oils for use herein include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following general formula (VIII):

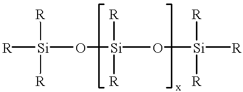

wherein R is aliphatic, preferably alkyl or alkenyl, or aryl, R can be substituted or unsubstituted, and x is an integer from 1 to about 8,000. Suitable unsubstituted R groups for use herein include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

The aliphatic or aryl groups substituted on the siloxane chain may have any structure so long as the resulting silicones remain fluid at room temperature, are hydrophobic, are neither irritating, toxic nor otherwise harmful when applied to the hair, are compatible with the other components of the hair bleaching compositions, are chemically stable under normal use and storage conditions, are insoluble in the hair bleaching compositions herein, and are capable of being deposited on and conditioning the hair. The two R groups on the silicon atom of each monomeric silicone unit may represent the same or different groups. Preferably, the two R groups represent the same group.

Preferred alkyl and alkenyl substituents are C₁ to C₅ alkyls and alkenyls, more preferably from C₁ to C₄, most preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, most preferably from C₁ to C₂. As discussed above, the R substituents can also contain amino functionalities (e.g. alkamino groups), which can be primary, secondary or tertiary amines or quaternary ammonium. These include mono-, di- and tri- alkylamino and alkoxyamino groups, wherein the aliphatic portion chain length is preferably as described above. The R substituents may also be substituted with other groups, such as halogens (e.g. chloride, fluoride, and bromide), halogenated aliphatic or aryl groups, hydroxy (e.g. hydroxy substituted aliphatic groups), and mixtures thereof. Suitable halogenated R groups could include, for example, tri-halogenated (preferably tri-fluoro) alkyl groups such as —R¹CF₃, wherein R¹ is a C₁-C₃ alkyl. An example of such a polysiloxane includes, but is not limited to, polymethyl 3,3,3-trifluoropropylsiloxane.

Suitable R groups for use herein include, but are not limited to: methyl, ethyl, propyl, phenyl, methylphenyl and phenylmethyl. Specific non-limiting examples of preferred silicones include: polydimethylsiloxane, polydiethylsiloxane, and polymethylphenylsiloxane. Polydimethyl-siloxane is especially preferred. Other suitable R groups include: methyl, methoxy, ethoxy, propoxy, and aryloxy. The three R groups on the end caps of the silicone may represent the same or different groups.

Non-volatile polyalkylsiloxane fluids that may be used include, for example, low molecular weight polydimethylsiloxanes. These siloxanes are available, for example, from the General Electric Company in their Viscasil R and SF 96 series, and from Dow Corning in their Dow Corning 200 series. Polyalkylaryl siloxane fluids that may be used also include, for example, polymethylphenylsiloxanes. These siloxanes are available, for example, from the General Electric Company as SF 1075 methyl phenyl fluid or from Dow Coming as 556 Cosmetic Grade Fluid. Polyether siloxane copolymers that may be used include, for example, a polypropylene oxide modified polydimethylsiloxane (e.g., Dow Coming DC-1248) although ethylene oxide or mixtures of ethylene oxide and propylene oxide may also be used. The ethylene oxide and polypropylene oxide concentrations must be sufficiently low to prevent solubility in water and the composition described herein.

Alkylamino substituted silicones suitable for use herein include, but are not limited to, those which conform to the following general formula (IX):

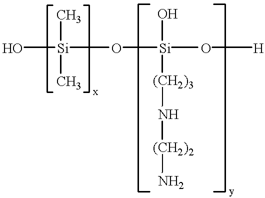

wherein x and y are integers. This polymer is also known as “amodimethicone.”

b. Cationic Silicones

Cationic silicone fluids suitable for use herein include, but are not limited to, those which conform to the general formula (X):

(R₁)ₐG₃₋ₐ—Si-(-OSiG₂)ₙ—(OSiG_{b}(R₁)_{2-b)m}—O—Sig₃₋ₐ(R₁)ₐ

wherein G is hydrogen, phenyl, hydroxy, or C₁-C₈ alkyl, preferably methyl; a is 0 or an integer having a value from 1 to 3, preferably 0; b is 0 or 1, preferably 1; n is a number from 0 to 1,999 preferably from 49 to 149; m is an integer from 1 to 2,000, preferably from 1 to 10; the sum of n and m is a number from 1 to 2,000, preferably from 50 to 150; R₁ is a monovalent radical conforming to the general formula CqH_{2q}L, wherein q is an integer having a value from 2 to 8 and L is selected from the following groups:

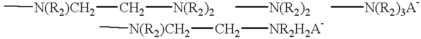

wherein R₂ is hydrogen, phenyl, benzyl, or a saturated hydrocarbon radical, preferably an alkyl radical from about C₁ to about C₂₀, and A⁻ is a halide ion.

An especially preferred cationic silicone corresponding to formula (X) is the polymer known as “trimethylsilylamodimethicone”, which is shown below in formula (XI):

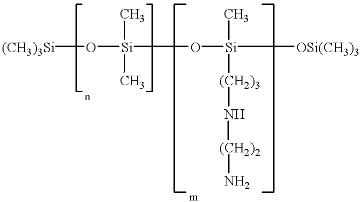

Other silicone cationic polymers which may be used herein are represented by the general formula (XII):

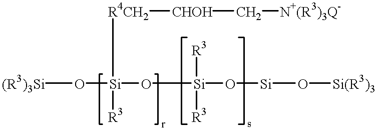

wherein R³ is a monovalent hydrocarbon radical from C₁ to C₁₈, preferably an alkyl or alkenyl radical, such as methyl; R4 is a hydrocarbon radical, preferably a C₁ to C₁₈ alkylene radical or a C₁ to C₁₈ alkyleneoxy radical, more preferably a C₁ to C₈ alkyleneoxy radical; Q is a halide ion, preferably chloride; r is an average statistical value from 2 to 20, preferably from 2 to 8; s is an average statistical value from 20 to 200, preferably from 20 to 50. A preferred polymer of this class is known as UCARE (RTM) SILICONE ALE 56, available from Union Carbide.

c. Silicone Gums

Other silicone fluids suitable for use herein are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, all of which descriptions are incorporated herein by reference. The silicone gums will typically have a weight average molecular weight in excess of about 200,000, preferably from about 200,000 to about 1,000,000. Specific non-limiting examples of silicone gums for use herein include polydimethylsiloxane, (polydimethylsiloxane) (methylvinylsiloxane) copolymer, poly(dimethylsiloxane) (diphenyl siloxane) (methylvinylsiloxane) copolymer and mixtures thereof.

d. High Refractive Index Silicones

Other non-volatile, insoluble silicone fluid conditioning agents that are suitable for use herein are those known as “high refractive index silicones,” having a refractive index of at least about 1.46, preferably at least about 1.48, more preferably at least about 1.52, most preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general Formula (VIII) above, as well as cyclic polysiloxanes such as those represented by Formula (XIII) below:

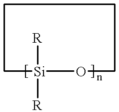

wherein R is as defined above, and n is a number from about 3 to about 7, preferably from about 3 to about5.

The high refractive index polysiloxane fluids contain an amount of aryl-containing R substituents sufficient to increase the refractive index to the desired level, which is described above. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted. Substituents include aliphatic substituents, and may also include alkoxy substituents, acyl substituents, ketones, halogens (e.g., C₁ and Br), amines, and the like. Examples of aryl-containing groups include, but are not limited to, sub-stituted and unsubstituted arenes, such as phenyl, and phenyl derivatives, such as phenyls with C₁-C₅ alkyl or alkenyl substituents. Specific non-limiting examples include: allylphenyl, methyl phenyl and ethyl phenyl, vinyl phenyls (e.g. styrenyl), and phenyl alkynes (e.g. phenyl C₂-C₄ alkynes). Heterocyclic aryl groups include, but are not limited to, substituents derived from furan, imidazole, pyrrole, pyridine, and the like. Examples of fused aryl ring substituents include, but are not limited to, napthalene, coumarin, and purine.

Generally, the high refractive index polysiloxane fluids will have a degree of aryl-containing substituents of at least about 15%, preferably at least about 20%, more preferably at least about 25%, even more preferably at least about 35%, most preferably at least about 50%. Typically, the degree of aryl substitution will be less than about 90%, more generally less than about 85%, preferably from about 55% to about 80%.

The high refractive index polysiloxane fluids are also characterized by relatively high surface tensions as a result of their aryl substitution. Generally, the polysiloxane fluids will have a surface tension of at least about 24 dynes/cm², typically at least about 27 dynes/cm². Surface tension, for purposes hereof, is measured by a de Nouy ring tensiometer according to Dow Corning Corporate Test Method CTM 0461 (Nov. 23, 1971). Changes in surface tension can be measured according to the above test method or according to ASTM Method D 1331.

Preferred high refractive index polysiloxane fluids have a combination of phenyl or phenyl derivative substituents (most preferably phenyl), with alkyl substituents, preferably C₁-C₄ alkyl (most preferably methyl), hydroxy, or C₁-C₄ alkylamino (especially -R¹NHR²NH2 wherein each R¹ and R² independently is a C₁-C₃ alkyl, alkenyl, and/or alkoxy). High refractive index polysiloxanes are available from Dow Coming, Huls America, and General Electric.

When high refractive index silicones are used herein, they are preferably used in solution with a spreading agent, such as a silicone resin or a surfactant, to reduce the surface tension by a sufficient amount to enhance spreading and thereby enhance the glossiness (subsequent to drying) of hair treated with the compositions. Generally, an amount of the spreading agent is used that is sufficient to reduce the surface tension of the high refractive index polysiloxane fluid by at least about 5%, preferably at least about 10%, more preferably at least about 15%, even more preferably at least about 20%, most preferably at least about 25%. Reductions in surface tension of the polysiloxane fluid/spreading agent mixture may improve shine of the hair.

Also, the spreading agent will preferably reduce the surface tension by at least about 2 dynes/cm², preferably at least about 3 dynes/cm², even more preferably at least about 4 dynes/cm², most preferably at least about 5 dynes/cm².

The surface tension of the mixture of the polysiloxane fluid and the spreading agent, at the proportions present in the final product, is preferably less than or equal to about 30 dynes/cm², more preferably less than or equal to about 28 dynes/cm2, most preferably less than or equal to about 25 dynes/cm². Typically, the surface tension will be in the range from about 15 dynes/cm² to about 30 dynes/cm, more typically from about 18 dynes/cm² to about 28 dynes/cm, and most generally from about 20 dynes/cm² to about 25 dynes/cm.

The weight ratio of the highly arylated polysiloxane fluid to the spreading agent will, in general, be from about 1000:1 to about 1:1, preferably from about 100:1 to about 2:1, more preferably from about 50:1 to about 2:1, most preferably from about 25:1 to about 2:1. When fluorinated surfactants are used, particularly high polysiloxane fluid to spreading agent ratios may be effective due to the efficiency of these surfactants. Thus, it is contemplated that ratios significantly above 1000:1 may be used.

Silicone fluids suitable for use herein are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984), all of which descriptions are incorporated herein by reference.

e. Silicone Resins

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin. As is apparent to one of ordinary skill in the art, the degree of cross-linking that is required in order to result in a silicone resin will vary according to the specific silane units incorporated into the silicone resin. Generally, silicone materials which have a sufficient level of trifunctional and tetrafunctional siloxane monomer units (and hence, a sufficient level of cross-linking) such that they dry down to a rigid, or hard, film are considered to be silicone resins. The ratio of oxygen atoms to silicon atoms is indicative of the level of cross-linking in a particular silicone material. Silicone resins suitable for use in the compositions of the present invention generally have at least about 1.1 oxygen atoms per silicon atom. Preferably, the ratio of oxygen to silicon atoms is at least about 1.2:1.0. Silanes used in the manufacture of silicone resins include, but are not limited to: monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are available from General Electric as GE SS4230 and GE SS4267. Commercially available silicone resins are generally supplied in a dissolved form in a low viscosity volatile or non-volatile silicone fluid. The silicone resins for use herein should be supplied and incorporated into the present compositions in such dissolved form, as will be readily apparent to one of ordinary skill in the art. Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence. Typical alternate substituents include, but are not limited to, groups such as vinyl, phenyls, amines, hydroxyls, and the like. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or M′ in a silicone resin indicates higher levels of cross-linking. As discussed above, however, the overall level of cross-linking can also be indicated by the oxygen to silicon ratio.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, is preferably from about 4:1 to about 400:1, more preferably from about 9:1 to about 200:1, most preferably from about 19:1 to about 100:1, particularly when the silicone fluid component is a polydimethylsiloxane fluid or a mixture of polydimethylsiloxane fluid and polydimethylsiloxane gum as described above. Insofar as the silicone resin forms a part of the same phase in the compositions hereof as the silicone fluid, i.e. the conditioning active, the sum of the fluid and resin should be included in determining the level of silicone conditioning agent in the composition.

2. Organic Conditioning Oils

The conditioning component of the compositions of the present invention may also comprise from about 0.05% to about 3%, by weight of the composition, preferably from about 0.08% to about 1.5%, more preferably from about 0.1% to about 1%, of at least one organic conditioning oil as the conditioning agent, either alone or in combination with other conditioning agents, such as the silicones (described above). The conditioning oils may add shine and luster to the hair. Additionally, they may enhance dry combing and dry hair feel.

The organic conditioning oils suitable for use as the conditioning agent herein are preferably low viscosity, water insoluble, liquids selected from the hydrocarbon oils, polyolefins, fatty esters, and mixtures thereof. The viscosity, as measured at 40° C., of such organic conditioning oils is preferably from about 1 centipoise to about 200 centipoise, more preferably from about 1 centipoise to about 100 centipoise, most preferably from about 2 centipoise to about 50 centipoise.

a. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation, another is VERSAGEL (RTM) ME 750 hydrogenated polyisobutene, available from Penreco.

b. Polyolefins

Organic conditioning oils for use in the compositions of the present invention can also include liquid polyolefins, more preferably liquid poly-α-olefins, most preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

c. Fatty Esters

Other suitable organic conditioning oils for use as the conditioning agent in the compositions of the present invention include, but are not limited to, fatty esters, typically having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Suitable for use in the compositions of the present invention are alkyl and alkenyl esters of fatty acids having from about C₁₀ to about C₂₂ aliphatic chains, and alkyl and alkenyl fatty alcohol carboxylic acid esters having a C₁₀ to about C₂₂ alkyl and/or alkenyl alcohol-derived aliphatic chain, and mixtures thereof. Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR, wherein R′ and R are alkyl or alkenyl radicals, and the sum of carbon atoms in R′ and R is at least 10, preferably at least 20. The mono-carboxylic acid ester need not necessarily contain at least one chain with at least 10 carbon atoms; rather the total number of aliphatic chain carbon atoms must be least 10. Specific non-limiting examples of mono-carboxylic acid esters include: isopropyl myristate, glycol stearate, and isopropyl laurate.

Still other fatty esters suitable for use in the compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, adipic acid, hexanoic acid, heptanoic acid, and octanoic acid). Specific non-limiting examples of di- and tri- alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxy-ethylene sorbitan fatty acid esters.

Still other fatty esters suitable for use in the compositions of the present invention are glycerides, including, but not limited to, mono-, di-, and tri-glycerides, preferably di- and tri-glycerides, most preferably triglycerides. For use in the compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters. Some preferred synthetic esters conform to the general formula (XIV):

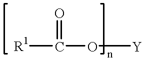

wherein R′ is a C₇ to C₉alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general formula (XV):

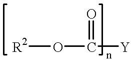

wherein R² is a C₈ to C₁₀ alkyl, alkenyl, hydroxyalkyl or hydroxyalkenyl group; preferably a saturated alkyl group, more preferably a saturated, linear, alkyl group; n and Y are as defined above in formula (XV).

Specific non-limiting examples of suitable synthetic fatty esters for use in the compositions of the present invention include: P43 (C₈-C₁₀ triester of trimethylolpropane), MCP-684 (tetraester of 3,3 diethanol-1,5 pentadiol), MCP 121 (C₉-C₁₀ diester of adipic acid), all of which are available from Mobil Chemical Company.

Also suitable for use as conditioning agents in the inventive compositions described herein are polyol fatty acid polyesters. A “polyol” is a polyhydric alcohol containing at least 4, preferably from 4 to 11 hydroxyl groups. A “polyol fatty acid polyester” is a polyol having at least 4 fatty acid ester groups. Typically, at least about 85%, of the hydroxyl groups of the polyol are esterified. In the case of sucrose polyesters, typically from 7 to 8 of the hydroxyl groups of the polyol are esterified. The polyol fatty acid esters typically contain C₄ to C₂₆ fatty acid radicals. A preferred sucrose polyester for use herein is olestra, sold under the trade name OLEAN (RTM), available from The Procter and Gamble Company. This oil, which is a blend of sucrose ester fatty acids (predominantly C₁₆ to C₁₈, and about 1% to about 2% C₁₄ to C₁₈), is described in U.S. Pat. Nos. 5,085,884, (Young, et al.) issued Feb. 4, 1992, and 5,422,131, (Elsen, et al.) issued Jun. 6, 1995, both of which descriptions are incorporated herein by reference.

3. Polyalkylene Glycol

The hair growth regulating compositions of the present invention may comprise from about 0.005% to about 1.5%, by weight of the composition preferably from about 0.025% to about 0.1%, more preferably from about 0.05% to about 1%, more preferably from about 0.1% to about 0.5%, most preferably from about 0.1% to about 0.3%, of selected polyalkylene glycols suitable for application to the hair or skin. Such polyalkylene glycols should be physically and chemically compatible with the essential components described herein, and should not otherwise unduly impair product stability, aesthetics, or performance.

The polyalkylene glycols suitable for use in the hair growth regulating compositions herein are characterized by the general Formula (XVI):

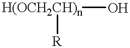

wherein R is hydrogen, methyl, or mixtures thereof, preferably hydrogen, and n is an integer having an average value from about 1,500 to about 120,000, preferably from about 1,500 to about 50,000, more preferably from about 2,500 to about 25,000, and most preferably from about 3,500 to about 15,000. When R is hydrogen, these materials are polymers of ethylene oxide, which are also known as polyethylene glycols. When R is methyl, these materials are polymers of propylene oxide, which are also known as polypropylene glycols. When R is methyl, it is also understood that various positional isomers of the resulting polymers can exist. Preferred for use herein are polyethylene glycols, polypropylene glycols, and mixtures thereof.

Specific non-limiting examples of polyethylene glycol polymers for use in the hair growth regulating compositions of the present invention include: PEG 2M, wherein R is hydrogen and n has an average value of about 2,000 (e.g. POLYOX WSR (RTM) N-10, available from Union Carbide); PEG SM, wherein R is hydrogen and n has an average value of about 5,000 (e.g. POLYOX WSR (RTM) N-35 and POLYOX WSR (RTM) N-80, both available from Union Carbide); PEG 7M, wherein R is hydrogen and n has an average value of about 7,000 (e.g. POLYOX WSR (RTM) N-750, available from Union Carbide); PEG 9M, wherein R is hydrogen and n has an average value of about 9,000 (e.g. POLYOX WSR (RTM) N-3333, available from Union Carbide); PEG 14 M, wherein R is hydrogen and n has an average value of about 14,000 (e.g. POLYOX WSR (RTM) N-3000, available from Union Carbide); PEG 23M, wherein R is hydrogen and n has an average value of about 23,000 (e.g. POLYOX WSR (RTM) N-12k, available from Union Carbide); PEG 90M, wherein R is hydrogen and n has an average value of about 90,000 (e.g. POLYOX WSR (RTM) 301, available from Union Carbide); and PEG 100M, wherein R is hydrogen and n has an average value of about 100,000 (e.g. CARBOWAX (RTM) PEG 4600, available from Union Carbide). Preferred polyethylene glycols include PEG 7M, PEG 14M, PEG 25M, PEG 90M, and mixtures thereof.

4. Other Conditioning Agents

Also suitable for use in the compositions herein are the conditioning agents described by the Procter & Gamble Company in U.S. Pat. Nos. 5,674,478, and 5,750,122, both of which are incorporated herein in their entirety by reference. Yet other conditioning agents suitable for use herein are the series of conditioners available from International Specialty Chemicals, such as the GAFQUAT (RTM) series of quaternary copolymers, and the ARQUAD (RTM) series of quaternary ammonium salts, available from Akzo Nobel. Also suitable for use herein are those conditioning agents described in U.S. Pat. Nos. 4,529,586 (Clairol), 4,507,280 (Clairol), 4,663,158 (Clairol), 4,197,865 (L'Oreal), 4,217,914 (L'Oreal), 4,381,919 (L'Oreal), and 4,422,853 (L'Oreal), all of which descriptions are incorporated herein by reference. Some other preferred silicone conditioning agents for use in the hair growth regulating compositions of the present invention include: ABIL (RTM) S201 (dimethicone/sodium PG-propyldimethicone thiosulfate copolymer), available from Goldschmidt; DC Q2-8220 (trimethylsilyl amodimethicone) available from Dow Coming; DC 949 (amodimethicone, cetrimonium chloride, and Trideceth-12), available from Dow Coming; DC 749 (cyclomethicone and trimethylsiloxysilicate), available from Dow Coming; DC2502 (cetyl dimethicone), available from Dow Coming; BC97/004 and BC 99/088 (amino functionalized silicone microemulsions), available from Basildon Chemicals; GE SME253 and SM2115-D2_and SM2658 and SF1708 (amino functionalized silicone microemulsions), available from General Electric; siliconized meadowfoam seed oil, available from Croda; and those silicone conditioning agents described by GAF Corp. in U.S. Pat. No. 4,834,767 (quaternized amino lactam), by Biosil Technologies in U.S. Pat. No. 5,854,319 (reactive silicone emulsions containing amino acids), and by Dow Corning in U.S. Pat. No. 4,898,585 (polysiloxanes), all of which descriptions are incorporated herein by reference.

Other preferred conditioning agents for use herein include quaternary species, such as QUADRISOFT (RTM) LM 200 (quatemized cellulose), available from Amerchol; Polymer KG30M (polyquaternium 10 and quaternized cellulose), INCROQUAT (RTM) behenyl trimonium methosulfate (cetearyl alcohol and behentrimonium methosulfate), available from Croda; MERQUAT (RTM) S (quaternary ammonium resin), available from Calgon; GAFQUAT (RTM) series 755 and 440 (cationic quaternized copolymers), available from ISP; AKYPOQUAT (RTM) 131, available from Kao; SALCARE (RTM) SC60 (quaternary ammonium resin), or SALCARE (RTM) SC95 or SC96 (cationic liquid dispersion thickeners), all available from Ciba; and MEADOWQUAT (RTM) HG (PEG-2-dimeadowfoamamido-ethylmonium methosulfate), available from Fanning.

Still yet other preferred conditioning agents for use herein include protein derivatives, such as CRODASONE (RTM) W (hydrolyzed wheat protein silicone copolymer) and HYDROTRITICUM (RTM) QM (quaternary hydrolyzed wheat protein), both available from Croda; and polymers, such as POLYOX (RTM) 60K and POLYOX (RTM) 10 (polyoxyethylenes), both available from Amerchol, and polyethyleneimines, available from BASF.

C. Cationic Polymer

The hair growth regulating compositions of the present invention may comprise from about 0.02% to about 5%, by weight of the composition, preferably from about 0.05% to about 3%, more preferably from about 0.1% to about 2%, most preferably from about 0.5% to about 1%, of at least one organic, cationic deposition and conditioning polymer suitable for application to the hair or skin. Such cationic polymers should be physically and chemically compatible with the essential components described herein, and should not otherwise unduly impair product stability, aesthetics or performance.

1. Physical Properties and Types of the Cationic Polymers

The cationic polymers useful in the present invention must be selected and must be present at a level such that the cationic polymers are soluble in the inventive compositions. The average molecular weight of cationic conditioning polymers suitable for use herein is typically from about 5,000 to about 10,000,000, preferably from about 100,000 to about 2,000,000, more preferably from about 200,000 to about 1,500,000, more preferably from about 250,000 to about 850,000, more preferably from about 350,000 to about 850,000, most preferably from about 350,000 to about 500,.000. The polymers have a cationic charge density typically from about 0.2 meq/g to about 7 meq/g, as measured at the pH of intended use of the compositions described herein, preferably from about 0.4meqlgm to about 5 meq/g, more preferably from about 0.6meq/g to about 2 meq/g, more preferably from about 0.5 meq/g to about 0.1 meq/g, more preferably from about 0.5 meq/g to about 0.9 meq/g. Any anionic counterions may be use in association with the cationic polymers so long as the cationic polymers remain soluble in the composition, and so long as the counterions are physically and chemically compatible with the essential components of the compositions described herein or do not otherwise unduly impair product performance, stability or aesthetics. Non-limiting examples of such counterions include: halides (e.g., chloride, fluoride, bromide, iodide), sulfate, methylsulfate, and mixtures thereof. Examples of cationic polymers which may be suitably employed in the hair bleaching compositions herein include, but are not limited to cationic polysaccharides (e.g. cationic cellulose derivatives and cationic guars), copolymers of vinyl monomers, vinyl pyrrolidone copolymers, cationic modified proteins, and certain polymeric quaternary salts. Such cationic polymers are described in detail below.

2. Cationic Polysaccharides

Preferred cationic polymers for use in the hair growth regulating compositions of the present invention are those known as cationic polysaccharides. Cationic polysaccharides are those polymers based on C₅ to C₆ sugars and derivatives which have been made cationic by engrafting of cationic moieties on the polysaccharide backbone, and include homopolymers, copolymers, terpolymers, and so forth, of quaternary ammonium or cationic amine-substituted monomer units, optionally in combination with non-cationic monomers. The polysaccharides may be composed of one type of sugar or of more than one type. The cationic amines can be primary, secondary, or ter-tiary amines (preferably secondary or tertiary), depending upon the particular species and the selected pH of the compositions described herein. The monomers may be in straight chain or branched chain geometric arrangements. All of the monomer units may have cationic nitrogen-containing moieties attached thereto, preferably some of the monomer units do not have such moieties attached. Non-limiting examples of cationic polysaccharides are described in the CTFA Cosmetic Ingredient Dictionary, 3d ed., edited by Estrin, Crosley, and Haynes, (The Cosmetic, Toiletry, and Fragrance Association, Inc., Washington, D.C. (1982), which description is incorporated herein by reference.

Cationic polysaccharide polymers include the following: cationic celluloses and hydroxyethylcelluloses; cationic starches and hydroxyalkyl starches; cationic polymers based on the galactomannan copolymer known as guar gum obtained from the endosperm of the guar bean; cationic polymers based on arabinose vegetable gums; cationic polymers derived from xylose polymers (such as those found in wood, straw, cottonseed hulls, and corn cobs); cationic polymers derived from fucose polymers (such as those found as a component of cell walls in seaweed); cationic polymers derived from fructose polymers (such as Inulin, which is found in certain plants); cationic polymers based on acid-containing sugars (such as galacturonic acid and glucouronic acid); cationic polymers based on amine sugars (such as galactosamine and glucosamine); cationic polymers based on 5 and 6 member ring polyalcohols; cationic polymers based on galactose monomers (such as those found in plant gums and mucilates); and cationic polymers based on mannose monomers (such as those found in plants, yeasts, and red algae). Preferred are cationic celluloses and hydroxyethylcelluloses; cationic starches and hydroxyalkyl starches; cationic polymers based on guar gum, and mixtures thereof.

a. Cationic Cellulose Derivatives

Suitable polysaccharide cationic polymers for use in the hair growth regulating compositions of the present invention are the cationic cellulose derivatives and cationic starch derivatives. Such cationic polymers include those which conform to the general Formula (XVII):

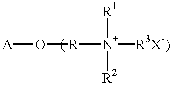

wherein A is an anhydroglucose residual group (e.g. a starch or cellulose anhydroglucose residual); R is an alkylene oxyalkylene, polyoxyalkylene, or hydroxyalkylene group, or combination thereof; R¹, R², and R³ are independently alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R¹, R², and R³) preferably being about 20 or less; and X is an anionic counterion as described above. Preferred cationic cellulose polymers include, but are not limited to, those polymers available from Amerchol Corporation, in their Polymer JR and LR series of polymers, as salts of hydroxyethyl cellulose reacted with trimethyl ammonium substituted epoxide, known in the industry (CTFA) as Polyquaternium 10 (e.g. JR 30M (RTM), available from Amerchol Corporation). Preferred Polyquaternium 10 polymers for use herein, typically have a charge density from about 0.3 meqlg to about 3 meq/g and a molecular weight from about 200,000 to about 1,500,00. Another non-limiting of a preferred type of cationic cellulose includes the poly-meric quaternary ammonium salt of hydroxyethyl cellulose reacted with lauryl dimethyl ammonium-substituted epoxide, known in the industry (CTFA) as Polyquaternium 24, (e.g. Polymer LM 200 (RTM), available from Amerchol Corporation).

Also suitable for use herein are those quaternary nitrogen-containing cellulose copolymers of hydroxyethylcellulose reacted with diallyldimethyl ammonium chloride, known in the industry (CTFA) as Polyquaternium 4 (e.g. CELQUAT (RTM) H-100, available from National Starch Corporation). Quaternary nitrogen-containing cellulose ethers suitable for use herein are described by the Procter & Gamble Company in U.S. Pat. No. 3,962,418, which is incorporated herein by reference in its entirety, and still other copolymers of etherified cellulose and starch suitable for use herein are described in by L'Oreal in U.S. Pat. No. 3,958,581, which description is incorporated herein by reference.

b. Cationic Guars

Other suitable polysaccharide cationic polymers for use in the hair growth regulating compositions of the present invention are cationic guar polymers. Guars are cationically substituted galactomannan (guar) gum derivatives. The molecular weight of such derivatives ranges typically from about 50,000 to about 2,500,000, preferably from about 50,000 to about 1,000,000, more preferably from about 50,000 to about 700,000.

Guar gum for use in preparing these guar gum derivatives is typically obtained as a naturally occurring material from the seeds of the guar plant. The guar molecule itself is a straight chain mannan branched at regular intervals with single membered galactose units on alternative mannose units. The mannose units are linked to each other by means of β (14) glycosidic linkages. The galactose branching arises by way of an α (1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide the requisite cationic charge density described above.

Suitable quaternary ammonium compounds for use in forming the cationic guar polymers include those conforming to the general formula (XVIII):

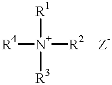

wherein where R¹, R² and R³ are methyl or ethyl groups; R⁴ is either an epoxyalkyl group of the general formula (XIX):

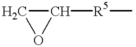

or R⁴ is a halohydrin group of the general Formula (XX):

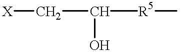

wherein R₅ is a C₁ to C₃ alkylene; X is chlorine or bromine, and Z is an anion such as Cl₃₁ , Br⁻, I³¹ or HSO₄⁻.

Cationic guar polymers (cationic derivatives of guar gum) formed from the reagents described above are represented by the general Formula (XXI):

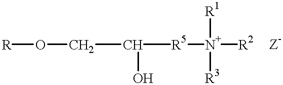

wherein R is guar gum. Preferably, the cationic guar polymer is guar hydroxypropyltrimethylammonium chloride, which can be more specifically represented by the general Formula (XXII):

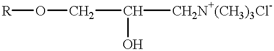

Specific non-limiting examples of cationic guar polymers which conform to Formula XXII include: JAGUAR (RTM) C 13S, having a cationic charge density of 0.8 meq/g (available from Rhodia Company) and JAGUAR (RTM) C 17, having a cationic charge density of 1.6meq/g (available from Rhodia Company). Other suitable cationic guar polymers include hydroxypropylated cationic guar derivatives. Still other suitable cationic polymers include copolymers of etherified guar, some examples of which are described in U.S. Pat. No. 3,958,581, which description is incorporated herein by reference.

3. Copolymers of Vinyl Monomers

Other suitable cationic polymers for use in the hair growth regulating compositions of the present invention are copolymers of vinyl monomers, having cationic protonated amine or quaternary ammonium functionalities, reacted with water soluble monomers. Non-limiting examples of such monomers include: acrylamide, methacrylamide, alkyl and dialkyl acrylamides, alkyl and dialkyl methacrylamides, alkyl acrylate, alkyl methacrylate, vinyl caprolactone, vinyl pyrrolidone, and mixtures thereof. The alkyl and dialkyl substituted monomers preferably have from C₁ to C₇ alkyl groups, more preferably from C₁ to C₃ alkyl groups. Other suitable monomers include vinyl esters, vinyl alcohol (made by hydrolysis of polyvinyl acetate), maleic anhydride, propylene glycol, ethylene glycol, and mixtures thereof.

Suitable cationic protonated amino and quaternary ammonium monomers, for inclusion in the cationic polymers of the hair bleaching composition herein, include vinyl compounds substituted with dialkylaminoalkyl acrylate, dialkylaminoalkyl methacrylate, monoalkylaminoalkyl acrylate, monoalkyl-aminoalkyl methacrylate, trialkyl methacryloxyalkyl ammonium salt, trialkyl acryloxyalkyl ammonium salt, diallyl quaternary ammonium salts; and vinyl quaternary ammonium monomers having cyclic cationic nitrogen-containing rings such as pyridinium, imidazolium, and quaternized pyrrolidones, such as alkyl vinyl imidazolium, alkyl vinyl pyridinium, and alkyl vinyl pyrrolidone salts. The alkyl portions of these monomers are preferably lower alkyls such as the C₁-C₃ alkyls.

Suitable amine-substituted vinyl monomers for use herein include, dialkylaminoalkyl acrylamide, and dialkylaminoalkyl methacrylamide, wherein the alkyl groups are preferably C₁ to C₇ hydro-carbyls, more preferably C₁ to C₃ alkyls.

4. Vinyl Pyrrolidone Copolymers

Other suitable cationic polymers for use in the hair growth regulating compositions of the present invention include: copolymers of 1-vinyl-2-pyrrolidone and 1-vinyl-3-methylimidazolium salt (e.g., chloride salt), known in the industry (CTFA) as Polyquaternium 16 (e.g. LUVIQUAT (RTM) FC 370, available from BASF Wyandotte Corporation); copolymers of 1-vinyl-2-pyrrolidone and dimethylaminoethyl methacrylate, known in the industry (CTFA) as Polyquaternium 11 (e.g. GAFQUAT (RTM) 755N, available from ISP Corporation); cationic diallyl quaternary ammonium-containing polymers, including, for example, dimethyldiallyl-ammonium chloride homopolymer, known in the industry (CTFA) as Polyquaternium 6; copolymers of acrylamide and dimethyldiallylammonium chloride, known in the industry (CTFA) as Polyquaternium 7; and mineral acid salts of amino-alkyl esters of homopolymers and copolymers of unsaturated C₃ to C₅ carboxylic acids, such as those described in U.S. Pat. No. 4,009,256, which description is incorporated herein by reference.

5. Cationic Modified Proteins and Polymeric Quaternary Salts

Still other cationic polymers for use in the hair growth regulating compositions of the present invention are cationic modified proteins, such as lauryldimonium hydroxypropyl collagen (e.g. CROQUAT (RTM) L, available from Croda Corporation), or cocodimonium hydroxypropyl hydrolized hair keratin (e.g. CROQUAT (RTM) HH, available from Croda Corporation). Other cationic polymers include the polymeric quaternary salt prepared the reaction of adipic acid and dimethylaminopropylamine, reacted with dichloroethyl ether, known in the industry (CTFA) as Polyquaternium 2 (e.g. MIRAPOL (RTM) AD-1, available from Rhodia), and the polymeric quaternary salt prepared by the reaction of azelaic acid and dimethylaminopropylether, known in the industry (CTFA) as Polyquaternium 18 (e.g. MIRAPOL (RTM) AZ-1, available from Rhodia Corporation).

6. Other Cationic Polymers

Suitable also for use herein are: Quaternium-19, Quaternium-23 , Quaternium-40, Quaternium-57, Poly(dipropyldiallylammonium chloride), Poly(methyl-.beta.-propaniodiallylammonium chloride), Poly(diallylpiperidinium chloride), Poly(vinyl pyridinium chloride), Quaternized poly(vinyl alcohol), Quaternized poly(dimethylaminoethylmethacrylate); and mixtures thereof.

D. Anti-dandruff Active

The hair growth regulating compositions of the present invention may, in some embodiments, comprise from about 0.1% to about 4%, by weight of the composition, preferably from about 0.1% to about 3%, most preferably from about 0.3% to about 2%, of an anti-dandruff active suitable for application to the hair or skin, which active may be particulate or soluble. The anti-dandruff active provides the compositions with anti-microbial activity. Such anti-dandruff actives should be physically and chemically compatible with the essential components of the composition, and should not otherwise unduly impair product stability, aesthetics or performance. Suitable, non4irniting examples of anti-dandruff particulate actives include: pyridinethione salts, selenium sulfide, particulate sulfur, and mixtures thereof. Preferred are pyridinethione salts. Soluble anti-dandruff actives are described below along with other anti-microbials.

1. Pyridinethione Salts

The hair growth regulating compositions of the present invention, may in some embodiments, comprise pyridinethione anti-dandruff particulates, especially 1-hydroxy-2-pyridinethione salts, are preferred particulate anti-dandruff agents for use. The concentration of pyridinethione anti-dandruff particulate typically ranges from about 0.1% to about 4%, by weight of the composition, preferably from about 0.1% to about 3%, most preferably from about 0.3% to about 2%. Preferred pyridinethione salts include those formed from heavy metals such as zinc, tin, cadmium, magnesium, aluminum and zirconium, preferably zinc, more preferably the zinc salt of 1-hydroxy-2-pyridinethione (known as “zinc pyridinethione” or “ZPT”), most preferably 1-hydroxy-2-pyridinethione salts in platelet particle form, wherein the particles have an average size of up to about 20μ, preferably up to about 5μ, most preferably up to about 2.5μ. Salts formed from other cations, such as sodium, may also be suitable. Pyridinethione anti-dandruff agents are described, for example, in U.S. Pat. Nos. 2,809,971; 3,236,733; 3,753,196; 3,761,418; 4,345,982; 4,323,683 4,379,753; and 4,470,982, all of which are incorporated herein by reference. It is contemplated, as noted below, that ZPT may also function as an agent that regulates hair growth as an activity enhancer.

2. Selenium Sulfide

Selenium sulfide is a particulate anti-dandruff agent suitable for use in the hair growth regulating compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x +y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. Nos. 2,694,668; 3,152,046; 4,089,945; and 4,885,107, all of which descriptions are incorporated herein by reference.

3. Sulfur

Sulfur may also be used as the particulate anti-dandruff agent in the hair growth regulating compositions of the present invention. Effective concentrations of the particulate sulfur are typically from about 1% to about 4%, by weight of the composition, preferably from about 2% to about 4%.

E. Activity Enhancer

The compositions herein may also optionally comprise in some embodiments one or more activity enhancers. Such agents can be chosen from a wide variety of molecules which can function in different ways to enhance the hair growth effects of a metal complex of an oxidized carbohydrate, of the present invention. Some activity enhancers may have a direct effect on the regulation of hair growth themselves, others may work synergistically with the metal complexes of oxidized carbohydrates of the present invention to affect the regulation of hair growth. Some activity enhancers can also function as vehicles for the metal complex of an oxidized carbohydrate, as noted above. It should be understood that the activity enhancers described below, while imparting some effect on the activity of the metal complexes of oxidized carbohydrates of the present invention, will continue to impart their commonly known effect to the compositions described herein, e.g. while an anti-microbial agent such as zinc pyrithione may affect the growth of hair, it will still impart a deleterious effect on microbes, such as those relating to dandruff. The activity enhancers, when present, are typically employed in the compositions herein at a level ranging from about 0.001% to about 15%, preferably from about 0.1% to about 10%, more preferably from about 0.5% to about 5% by weight of the composition. Non-limiting examples of activity enhancers are described below.

1. Vasodilators

Optional activity enhancers suitable for use herein include vasodilators, such as potassium channel agonists, including, for example, minoxidil and minoxidil derivatives, such as aminexil, and such as those described in U.S. Pat. Nos. 3,382,247, and 5,756,092, issued May 26, 1998, U.S. Pat. No. 5,772,990, issued Jun. 30, 1998, U.S. Pat. No. 5,760,043, issued Jun. 2, 1998, U.S. Pat. No. 328,914, issued Jul. 12, 1994, U.S. Pat. No. 5,466,694, issued Nov. 14, 1995, 5,438,058, issued Aug. 1, 1995, and U.S. Pat. No. 4,973,474, issued Nov. 27, 1990, and also minoxidil glucuronides, as described by Unilever in EP-A-0 242 967, minoxidil sulphates, as described by The Upjohn Co. in WO-A-86/04231, (all of which are herein incorporated by reference), and cromakalin and diazoxide can be used as optional hair growth regulating agents in the compositions herein. 2. Anti-androgens

Optional activity enhancers suitable for use herein include anti-androgens. Examples of suitable anti-androgens may include, but are not limited 5-oc-reductase inhibitors such as finasteride and those described in U.S. Pat, No. 5,516,779, issued May 14, 1996 (herein incorporated by reference) and in Nnane et al, Cancer Research 58, “Effects of Some Novel Inhibitors of C17,20-Lyase and 5α-Reductase in Vitro and in Vivo and Their Potential Role in the Treatment of Prostate Cancer., as well as cyproterone acetate, azelaic acid and its derivatives and those compounds described in U.S. Pat. No. 5,480,913, issued Jan. 2, 1996, flutamide, and those described in U.S. Pat. No. 5,411,981, issued May 2, 1995, U.S. Pat. No. 5,565,467, issued Oct. 15, 1996 and U.S. Pat. No. 4,910,226, issued Mar. 20, 1990, all of which are herein incorporated by reference.

3. Immunosuppressants

Optional activity enhancers suitable for use herein include imrnunosuppressants such as 1) cyclosporin and cyclosporin analogs including those described in U.S. Provisional Patent Application No. 60/122,925, Fulmer et al., “Method of Treating Hair Loss Using Non-Immunosuppressive Compounds”, filed Mar. 5, 1999, herein incorporated by reference, and 2) FK506 analogs such as those described in U.S. Provisional Patent Application No. 60/102,449, McIver et al., “Heterocyclic 2-Substituted Ketoamides”, filed Sep. 30, 1998, U.S. Provisional Patent Application No. 60/102,448, McIver et al., “2-Substituted Ketoamides”, filed Sep. 30, 1998, U.S. Provisional Patent Application No. 60/102,539, McIver et al., “2-Substituted Heterocyclic Sulfonamides”, filed Sep. 30, 1998, U.S. Provisional Patent Application No. 60/102,458, Tiesman et al., “Method of Treating Hair Loss Using Ketoamides”, filed Sep. 30, 1998, and U.S. Provisional Patent Application No. 60/102,437, McIver et al., “Method of Treating Hair Loss Using Sulfonamides”, filed Sep. 30, 1998, all of which are herein incorporated by reference.

4. Anti-microbial and Anti-fungal Actives

Optional activity enhancers suitable for use herein include anti-microbial and anti-fungal actives. Examples of anti-microbial and anti-fungal actives useful herein include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, clotrimazole, cetylpyridinium chloride (CPC), piroctone olamine, selenium sulfide, ketoconazole, triclocarbon, triclosan, zinc pyrithione, itraconazole, asiatic acid, hinokitiol, mipirocin and those described in EPA 0,680,745 (herein incorporated by reference), clinacycin hydrochloride, benzoyl peroxide, benzyl peroxide, minocyclin, phenoxy isopropanol, and mixtures thereof.

5. Anti-inflammatories

Anti-inflammatories can also be incorporated into the compositions herein as an optional activity enhancer. Examples of suitable anti-inflammatories may include glucocorticoids such as hydrocortisone, mometasone furoate and prednisolone, nonsteroidal anti-inflammatories including cyclooxygenase or lipoxygenase inhibitors such as those described in U.S. Pat. No. 5,756,092, and benzydamine, salicylic acid, those compounds described in EPA 0,770,399, published May 2, 1997, WO 94/06434, published Mar. 31, 1994 and FR 2,268,523, published Nov. 21, 1975, all of which are herein incorporated by reference, aspirin, ibuprofen, naproxen, indomethacin, piroxicam, flurbiprofen, meclofenamate sodium, ketoprofen, tenidap, tebufelone, and ketorolac.

6. Thyroid Hormones

Optional activity enhancers suitable for use herein include thyroid hormones and derivatives and analogs thereof. Examples of suitable thyroid hormones for use herein may include triiodothyrionine. Examples of thyroid hormone analogs which may be suitable for use herein include those described in U.S. Provisional Patent Application No. 60/136,996, Zhang et al., “Method of Treating Hair Loss”, filed Jun. 1, 1999, U.S. Provisional Patent Application No. 60/137,024, Zhang et al., “Method of Treating Hair Loss Using Biphenyl Compounds”, filed Jun. 1, 1999, U.S. Provisional Patent Application No. 60/137,022, Zhang et al., “Method of Treating Hair Loss Using Carboxyl Derivatives”, filed Jun. 1, 1999, U.S. Provisional Patent Application No. 60/137,023, Zhang et al., “Method of Treating Hair Loss Using Sulfonyl Thyromimetic Compounds”, filed Jun. 1, 1999, U.S. Provisional Patent Application No. 60/137,052, Youngquist et al., “Biaryl Compounds”, filed Jun. 1, 1999, U.S. Provisional Patent Application No. 60/137,063, Youngquist et al., “Sulfur-Bridged Compounds”, filed Jun. 1, 1999, and U.S. Provisional Patent Application No. 60/136,958, Youngquist et al., “Substituted Biaryl Ether Compounds”, filed Jun. 1, 1999.

7. Prostaglandin Agonists

Prostaglandin agonists (also known as “antagonists”) can also be used as optional activity enhancers in the compositions herein. Examples of suitable prostaglandins agonists or antagonists include latanoprost and those described in WO 98/33497, Johnstone, published Aug. 6, 1998, WO 95/11003, Stjernschantz, published Apr. 27, 1995, JP 97-100091, and Ueno, JP 96-134242, Nakamura.

8. Retinoids

Optional activity enhancers suitable for use herein include retinoids. Suitable retinoids may include isotretinoin, acitretin, and tazarotene.

9. Other Activity Enhancers

Suitable for use herein are: (i) nicotinic acid and esters thereof, particularly benzyl, nicotinate, methyl nicotinate and ethyl nicotinate; (ii) Panthenol (iii) alpha-1,4 esterified disaccharides as described by Choay S. A. in EP-A-0 064 012, (iv) Oligosaccharide derivatives, as described by Unilever in EP-A-0 211 610, (v) Proteoglycanase inhibitors, glycosarninoglycan chain cellular uptake inhibitors and glycosaminoglycanase inhibitor other than those disclosed herein, as described by Unilever in EP-A-0 277 428, (vi) Ethylenediaminetetraacetic acid or salts thereof, as described by Redken Laboratories, Inc. in U.S. Pat. No. 4,814,351, (vii) Esters of pyroglutamic acid, as described by Lever Brothers Company in U.S. Pat. No. 4,774,255, especially: pyroglutamic acid n-hexyl ester, pyroglutamic acid n-octyl ester, ethyl-2-[pyroglutamoyloxy]-n-propionate, linoleyl-2-[pyroglutamoyloxy]-n-caprylate, lauryl-2-[pyroglutamoyloxy]-n-caprylate, stearyl-2-[pyrogluta-moyloxy]-n-caprylate, glyceryl mono(2-[pyroglutamoyloxy]-n-propionate), glyceryl mono(2-[pyroglutamoyloxy]-n-caprylate), and glyceryl di(2-[pyroglutamoyloxy]-n-propionate); (viii) Aryl-substituted ethylenes, as described by Unilever in EP-A-0 403 238, (ix) Mono N-acylated amino acids, as described by Unilever in EP-A-0 415 598, especially: N-acetyl glycine (x) Saturated or unsaturated aliphatic alcohols having an odd number of carbon atoms of from 3 to 25 in number, especially: n-nonyl alcohol, (xi) Saturated or unsaturated aliphatic carboxylic acids having an odd number of carbon atoms of from 3 to 25 in number, especially: nonanoic acid; and (xii) mixtures thereof.

Still other activity enhancers suitable for use herein include flavinoids, ascomycin derivatives and analogs, histamine antagonists such as diphenhydramine hydrochloride, other triterpenes such as oleanolic acid and ursolic acid and those described in U.S. Pat. No. 5,529,769, JP 10017431, WO 95/35103, U.S. Pat. No. 5,468,888, JP 09067253, WO 92/09262, JP 62093215, U.S. Pat. No. 5,631,282, U.S. Pat. No. 5,679,705, JP 08193094, saponins such as those described in EP 0,558,509 to Bonte et al, published Sep. 8, 1993 and WO 97/01346 to Bonte et al, published Jan. 16, 1997 (both of which are herein incorporated by reference in their entirety), proeoglycanase or glycosaminoglycanase inhibitors such as those described in U.S. Pat. No. 5,015,470, issued May 14, 1991, U.S. Pat. No. 5,300,284, issued Apr. 5, 1994 and U.S. Pat. No. 5,185,325, issued Feb. 9, 1993 (all of which are herein incorporated in their entirety by reference) estrogen agonists and antagonists, pseudoterins, cytokine and growth factor promoters, analogs or inhibitors such as interleukin-1 inhibitors, interleukin-6 inhibitors, interleukin-10 promoters, and tumor necrosis factor inhibitors, vitamins such as vitamin D analogs and parathyroid hormone antagonists, Vitamin B12 analogs and pantheon, interfuron agonists and antagonists, hydroxyacids such as those described in U.S. Pat. No. 5,550,158, benzophenones and hydantoin anticonvulsants such as phenytoin.

Other activity enhancers are described in detail in, for example, JP 09-157,139 to Tsuji et al, published Jun. 17, 1997; EP 0277455 A1 to Mirabeau, published Aug. 10, 1988; WO 97/05887 to Cabo Soler et al, published Feb. 20, 1997; WO 92/16186 to Bonte et al, published Mar. 13, 1992; JP 62-93215 to Okazaki et al, published Apr.28, 1987; U.S. Pat. No. 4,987,150 to Kurono et al, issued Jan. 22, 1991; JP 290811 to Ohba et al, published Oct. 15, 1992; JP 05-286,835 to Tanaka et al, published Nov. 2, 1993, FR 2,723,313 to Greff, published Aug. 2, 1994, U.S. Pat. No. 5,015,470 to Gibson, issued May 14, 1991, U.S. Pat. No. 5,559,092, issued Sep. 24, 1996, U.S. Pat. No. 5,536,751, issued Jul. 16, 1996, U.S. Pat. No. 5,714,515, issued Feb. 3, 1998, EPA 0,319,991, published Jun. 14, 1989, EPA 0,357,630, published Oct. 6, 1988, EPA 0,573,253, published Dec. 8, 1993, JP 61-260010, published Nov. 18, 1986, U.S. Pat. No. 5,772,990, issued Jun. 30, 1998, U.S. Pat. No. 5,053,410, issued Oct. 1, 1991, and U.S. Pat. No. 4,761,401, issued Aug. 2, 1988, all of which are herein incorporated by reference.

10. Preferred Activity Enhancers

Some preferred activity enhancers for use in the hair growth regulating compositions of the present invention include: zinc salts of carboxylic acids, minoxidil, finasteride, cyclosporin, ketoconazole, triclocarbon, triclosan, zinc pyrithione, itraconazole, hinokitiol, mipirocin, hydrocortisone, tenidap, triiodothyrionine, latanoprost, isotretinoin, acitretin, tazarotene, nicotinic acid, niacinamide, glycosaminoglycanase inhibitors, ethylenediaminetetraacetic acid, oleanolic acid, ursolic acid, interleukin-1 inhibitors, interleukin-6 inhibitors, interleukin-10 promotors, saponins, triterpenes, betulinic acid, betulonic acid, crataegolic acid, celastrol, asiatic acid, inhibitors of 5-α-reductase, progesterone, 1,4-methyl-4-azasteroids, 17-β-N,N-diethylcarbamoyl4-methyl4-aza-5-α-androstan-3-one, androgen receptor antagonists, cyproterone acetate, azelaic acid, diazoxide, potassium channel openers, cromakalin, phenytoin, dutasteride, coal tar, zinc gluconate, manganese gluconate, glucocortisoids, macrolides, aminexil, ginkgo biloba, ivy, methyl salicylate, clinacycin hydrochloride, benzoyl peroxide, benzyl peroxide, minocyclin, and mixtures thereof.

F. Penetration Enhancers

The compositions according to the invention may also optionally comprise one or more penetration enhancers, which may potentiate the benefit of the metal complex of an oxidized carbohydrate by improving its delivery through the stratum corneum to its site of action in the immediate environment of the hair follicle close to the hair bulb.

Non-limiting examples of penetration enhancers which may be used herein include, for example: 2-methyl propan-2-ol; propan-2-ol; 1-propan-1,2-diol; ethyl-2-hydroxypropanoate; hexan-2,5-diol; POE(2) ethyl ether; di(2-hydroxypropyl) ether; pentan-2,4-diol; acetone; POE(2) methyl ether; 2-hydroxypropionic acid; 2-hydroxypropanoic acid; 2-hydroxyoctanoic acid; propan-1-ol; 1,4-dioxane; tetrahydrofuran; butan-1,4-diol; propylene glycol dipelargonate; polyoxypropylene 15 stearyl ether; octyl alcohol; POE ester of oleyl alcohol; oleyl alcohol; lauryl alcohol; dioctyl adipate; dicapryl adipate; di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, iso-propyl palmitate, ethyl laurate, 2-ethyl-hexyl pelargonate, iso-propyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hyroxyoctanoic acid, methylsulfoxide, N,N-dimethyl acetamide, N,N-dimethyl formamide, 2-pyrrolidone, 1-methyl-2-pyrrolidone, 5-methyl-2-pyrrolidone, 1,5-dirnethyl-2-pyrrolidone, 1-ethyl-2-pyrrolidone, phosphine oxides, sugar esters, tetrahydrofurfural alcohol, urea, diethyl-m-toluamide, l-dodecylazacyloheptan-2-one and those described in U.S. Pat. No. 5,015,470, issued May 14, 1991 and U.S. Pat. No. 5,496,827, issued Jul. 15, 1994 (both of which are herein incorporated in its entirety by reference), dimethylsulfonyl oxide (DMSO), octonol, niacinamide, and mixtures thereof.

G. Non-oxidative and Other Dyes

The hair growth regulating compositions of the present invention may, in some embodiment, include non-oxidative and other dye materials. Such non-oxidative and other dyes suitable for use in the hair growth regulating compositions and processes according to the present invention include semi-permanent, temporary and other dyes. Non-oxidative dyes as defined herein include the so-called ‘direct action dyes’, metallic dyes, metal chelate dyes, fibre reactive dyes and other synthetic and natural dyes. Various types of non-oxidative dyes are detailed in: Chemical and Physical Behaviour of Human Hair 3rd ed. Clarence Robbins (at pages 250-259); The Chemistry and Manufacture of Cosmetics. vol. IV. 2nd ed. Maison G. De Navarre (at chapter 45 by G. S. Kass (at pages 841-920)); Cosmetics: Science and Technology 2nd ed., vol. 11 Balsam Sagarin, (at chapter 23 by F. E. Wall (at pages 279-343)); The Science of Hair Care, edited by C. Zviak (at pages 235-261) and Hair Dyes, J. C. Johnson, Noyes Data Corp., Park Ridge, U.S.A. (1973), (at pages 3-91 and 113-139). Other dyes or pigments among those useful herein include those described in Handbook of Pharmaceutical Excipients, Second Edition pp. 126-134, 1994 by the American Pharmaceutical Association & the Pharmaceutical Press, incorporated by reference herein.

H. Suspending Agent

The hair growth regulating compositions of the present invention may, in some embodiments, comprise from about 0.1% to about 10%, by weight of the composition, preferably from about 0.3% to about 5%, more preferably from about 0.3% to about 2.5%, of a suspending agent suitable for application to the hair or skin. It is believed that the suspending agent suspends water-insoluble, dispersed materials in the shampoo compositions. Such suspending agent should be physically and chemically compatible with the essential components of the composition, and should not otherwise unduly impair product stability, aesthetics or performance. Examples of suspending agents which may be suitably employed in the compositions herein include, but are not limited to: acyl derivatives, long chain amine oxides, xanthan gum, and mixtures thereof. These and other suitable suspending agents are described in further detail below.

1. Acyl Derivatives and Long Chain Amine Oxides

Acyl derivative suspending agents include, but are not limited to: glyceryl esters, long chain hydrocarbyls, long chain esters of long chain fatty acids, long chain esters of long chain alkanol amides. Another suitable suspending agent group includes the long chain amine oxides. Acyl derivative and long chain amine oxide suspending agents are described in U.S. Pat. No. 4,741,855, which description is incorporated herein by reference.

Preferred acyl derivative suspending agents for use herein are glyceryl esters, which include C₁₆ to C₂₂ ethylene glycol esters of fatty acids. More preferred are the ethylene glycol stearates, both mono- and di-stearate, most preferred is ethylene glycol di-stearate containing less than about 7% of the mono-stearate.

Also suitable for use in the compositions herein are long chain (i.e. C₈ to C₂₂) hydrocarbyls, which include N,N-dihydrocarbyl amido benzoic acid and soluble salts thereof (e.g., Na, K), particularly N,N-di-(hydrogenated) C₁₆, C₁₈ and tallow amido benzoic acid species of this family, available from Stepan Company. Non-limiting examples of long chain esters of long chain fatty acids include: stearyl stearate and cetyl palmitate. Non-limiting examples of long chain esters of long chain alkanol amides include: stearamide diethanolamide distearate and stearamide monoethanolamide stearate. Non-limiting examples of suitable long chain amine oxides for use as suspending agents herein include the alkyl (C₁₆-C₂₂) dimethyl amine oxides (e.g. stearyl dimethyl amine oxide).

2. Xanthan Gum

Also suitable as a suspending agent herein is xanthan gum. The concentration of xanthan gum will typically range from about 0.1% to about 3%, by weight of the composition, preferably from about 0.4% to about 1.2%. The use of xanthan gum as a suspending agent in silicone containing shampoo compositions is described, for example, in U.S. Pat. No. 4,788,006, which description is incorporated herein by reference. Combinations of long chain acyl derivatives and xanthan gum may also be used as a suspending agent in the compositions of the present invention. Their use is described in U.S. Pat. No. 4,704,272, which description is incorporated herein by reference.

3. Other Suspending Agents

Still other suitable suspending agents for use in the hair growth regulating compositions of the present invention include carboxyvinyl polymers. Preferred among these polymers are the copolymers of acrylic acid crosslinked with polyallylsucrose, as described in U.S. Pat. No. 2,798,053, which description is incorporated herein by reference. Examples of these polymers include Carbopol 934, 940, 941, and 956, available from B. F. Goodrich Company.

Other suitable suspending agents for use herein include primary amines having a fatty alkyl moiety having at least about 16 carbon atoms (e.g. palmitamine, and stearamine), and secondary amines having two fatty alkyl moieties each having at least about 12 carbon atoms (e.g. dipalmitoylamine, and di-(hydrogenated tallow)-amine). Also suitable are di-(hydrogenated tallow)-phthalic acid amide, and cross-linked maleic anhydride-methyl vinyl ether copolymer. Still other suitable suspending agents may be used in the inventive compositions, including those that can impart a gel-like viscosity to the composition, such as water soluble or colloidally water soluble polymers like cellulose ethers (e.g., methylcellulose, hydroxybutyl methylcellulose, hydroxypropylcellulose, hydroxypropyl methylcellulose, hydroxyethyl ethylcellulose and hydroxyethylcellulose), guar gum, polyvinyl alcohol, polyvinyl pyrrolidone, hydroxypropyl guar gum, starch and starch derivatives, and other thickeners, viscosity modifiers, gelling agents, and mixtures thereof. A preferred viscosity modifier useful as a suspending agent is trihydroxystearin, (e.g. THIXN (RTM) R, available from Rheox Company).

I. Non-Steroidal Anti-Inflammatory Actives (NSAIDS)

Examples of NSAIDS suitable for use herein include the following categories: propionic acid derivatives; acetic acid derivatives; fenamic acid derivatives; biphenylcarboxylic acid derivatives; and oxicams. All of these NSAIDS are fully described in U.S. Pat. No. 4,985,459 to Sunshine et al., issued Jan. 15, 1991, incorporated by reference herein in its entirety. Examples of useful NSAIDS include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid.

J. Topical Anesthetics

Examples of topical anesthetic drugs suitable for use herein include benzocaine, lidocaine, bupivacaine, chlorprocaine, dibucaine, etidocaine, mepivacaine, tetracaine, dyclonine, hexyl-caine, procaine, cocaine, ketamine, pramoxine, phenol, and pharmaceutically acceptable salts thereof.

K. Sunscreen Actives

Also useful herein are sunscreening actives. A wide variety of sunscreening agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VII, pages 189 et seq., of Cosmetics Science and Technology, all of which are incorporated herein by reference in their entirety. Non-limiting examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991; these two references are incorporated by reference herein in their entirety. Especially preferred examples of these sunscreens include those selected from the group consisting of 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N- (2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof. Exact amounts of sunscreens which can be employed will vary depending upon the sunscreen chosen and the desired Sun Protection Factor (SPF) to be achieved. SPF is a commonly used measure of photoprotection of a sunscreen against erythema. See Federal Register, Vol. 43, No. 166, pp. 38206-38269, August 25, 1978, which is incorporated herein by reference in its entirety.

L. Flavoring Agents

Flavoring agents among those useful herein, particularly when the compositions of the present invention are to be administered orally, include those described in Remington's Pharnaceutical Sciences, 18th Edition, Mack Publishing Company, 1990, pp. 1288-1300, incorporated by reference herein.

M. Preservatives

Preferred preservatives include, but are not limited to, DMDM hydantoin, phenol, alkyl esters of parahydroxybenzoic acid, benzoic acid and the salts thereof, boric acid and the thereof, sorbic acid and the salts thereof, chorbutanol, benzyl alcohol, thimerosal, phenylmercuric acetate and nitrate, nitromersol, benzalkonium chloride, cetylpyridinium chloride, methyl paraben, propyl paraben, Germall 115, methyl, ethyl, propyl and butyl esters of hydroxybenzoic acid, EDTA, Euxyl (RTM) K400, and natural preservatives, such as benzyl alcohol, potassium sorbate and bisabalol, benzoic acid, sodium benzoate, and 2-phenoxyethanol. Particularly preferred are DMDM hydantoin, the salts of benzoic acid, benzalkonium chloride, methyl paraben and propyl paraben.

N. Sweeteners

Preferred sweeteners include, but are not limited to, sucrose, glucose, saccharin, and aspartame. Particularly preferred are sucrose and saccharin.

O. Other Optional Ingredients

The hair growth regulating compositions of the present invention may, in some embodiments, further comprise additional optional components known or otherwise effective for use in hair care or personal care products. The concentration of such optional ingredients generally ranges from zero to about 25%, more typically from about 0.05% to about 25%, even more typically from about 0.1% to about 15%, by weight of the composition. Such optional components should also be physically and chemically compatible with the essential components described herein, and should not otherwise unduly impair product stability, aesthetics or performance.

Non limiting examples of optional components for use in the shampoo composition include anti-static agents (e.g. tricetyl methyl ammonium chloride), foam boosters (e.g. fatty ester (e.g. C8-C₂₂) mono- and di (C1-C₅, especially C1-C₃) alkanol amides, preferably coconut monoethanolamide, coconut diethanolamide, and mixtures thereof), viscosity modifiers and thickeners (e.g. sodium chloride, sodium sulfate, and magnesium sulfate), pH adjusting agents (e.g. sodium citrate, citric acid, succinic acid, phosphoric acid, sodium hydroxide, and sodium carbonate), dyes, organic solvents or diluents, pearlescent aids, perfumes, fatty alcohols, proteins, skin active agents, vitamins, abrasives, absorbents, anti-caking agents, anti-oxidants (e.g. sodium sulphite, hydroquinone, sodium bisulphite, sodium metabisulphite and thyoglycolic acid, sodium dithionite, erythrobic acid and other mercaptans, which such anti-oxidants may be delivered using encapsulation techniques described in U.S. Pat. No. 5,053,051 (Goldwell), which description is incorporated herein by reference), biological additives, bulking agents, chelating agents, chemical additives, colorants, cosmetic astringents, cosmetic biocides, denaturants, drug astringents, external analgesics, film formers, opacifying agents, reducing agents, skin bleaching agents, and moisturizing agents (e.g. hyaluronic acid, chitin, and starch-grafted sodium polyacrylates such as SANWET (RTM) IM-1000, IM-1500 and IM-2500 available from Celanese Superabsorbent Materials, Portsmith, Va., USA, and described in U.S. Pat. No. 4,076,663, which description is incorporated herein by reference). Furthermore, the CTFA International Cosmetic Ingredient Dictionary and Handbook, 8^{th} ed., 1999, which is incorporated by reference herein in its entirety, describes a wide variety of non-limiting cosmetic and pharmaceutical ingredients commonly used in the hair and skin care industry, which are suitable for use in the compositions of the present invention.

### Methods of Manufacture

The metal complexes of oxidized carbohydrates of the present invention may be synthesized using any conventional method. Some typical methods are: (a) multiple synthesis method, as described in “The Synthesis and Analysis of Copper (II) Carboxylates,” Yoder, et al., Journal of Chemical Education (1995), vol. 72, pages 267 et seq.; (b) bivalent metal oxide method, as described in “Complex Formation Between D-lactobionate and Bivalent Metal Ions,” Frutos, et al., Canadian Journal of Chemistry, (1977), vol. 75, pages 405 - 413; and (c) ion exchange method, as described “Preparation of Zinc Gluconate by Ion Exchange Resin”, Dy, et al., Zhongguo Yiyao Gongye Zazhi Journal, (1992), vol. 23(4), page 156; all of which descriptions are incorporated herein by reference.

The compositions containing the metal complexes of oxidized carbohydrates of the present invention may be the form of a tablet, capsule, caplet, cream, gel, hydrogel, foam, mousse, liquid, solid, powder, tonic, rinse, shampoo, spray, paste, or other suitable form.

Alternatively, the compositions according to the present invention can be packaged in a kit, as follows: one article of the kit comprises an individually packaged component containing at least one metal complex of an oxidized carbohydrate, while further kit articles could comprise one or more components for use in pre-treatment or post-treatment steps as part of a regimen for regulating hair growth regulating, for further enhancing hair growth performance, for creating special and individualized hair growth regulation effects, or for addressing specific needs of the consumer.

### Methods of Use

The method of the present invention involves the administration of the compositions described herein for regulating hair growth in mammals (e.g., humans and domestic animals). It is also contemplated that the compositions may be administered to the skin for achieving skin benefits, and to the finger nails or toe nails for nail growth benefits.

The compositions of the present invention can be administered topically, orally or parenterally. A preferred method of using the present invention involves the topical application of the compositions described herein to the scalp, skin, and/or hair, more preferably to the scalp, skin, and/or hair where the scalp is already bald or balding. The amount of the composition and the frequency of application to scalp, skin, and/or hair can vary widely, depending on the desired effect and/or personal needs. Typically the composition is applied from about 1 to about 10 times per day, more typically from about 1 to about 6 times per day and most typically from 1 to 3 times per day. The compositions of the present invention can be also be used as a pre-treatment or post-treatment step to additional hair growth regulating processes taking place in order to further enhance hair growth performance or to create special and individualized hair growth regulation effects or to address specific needs of the consumer.

The topical compositions can be delivered the hair/scalp/skin from a variety of delivery devices. For example, the compositions can be incorporated into a medicated cleansing pad. Preferably these pads comprise form about 50% to about 75% of a substrate and from about 25% to about 50% of a liquid composition deliverable from the substrate. Suitable pads are described, for example, in U.S. Pat. No. 4,891,228; Thurman et al.; issued Jan. 2, 1990; and U.S. Pat. No. 4,891,227; Thaman et al.; issued Jan. 2, 1990, both of which are incorporated by reference. Alternatively, the compositions useful herein can be incorporated into and delivered from a soft-tipped or flexible dispensing device. These devices are useful for the controlled delivery of the compositions to the skin surface and have the advantage that the treatment composition itself never need be directly handled by the user. Non-limiting examples of these devices comprise a fluid container including a mouth, an applicator, means for holding the applicator in the mouth of the container and a normally closed pressure-responsive valve for permitting the flow of fluid from the container to the applicator upon the application of pressure to the valve. The fluid preferably contains from about 0.01% to about 20% of at least one metal complex of an oxidized carbohydrate, preferably from about 0.1% to about 10%, more preferably from about 1% to about 5%.

The valve can include a diaphragm formed from an elastically fluid impermeable material with a plurality of non-intersecting arcuate slits therein, where each slit has a base which is intersected by at least one other slit, and where each slit is out of intersecting relation with its own base, and wherein there is a means for disposing the valve in the container inside of the applicator. Examples of these applicator devices are described in U.S. Pat. Nos. 4,693,623 to Schwartzman; issued Sep. 25, 1987; 3,669,323; Harker et al.; issued Jun. 13, 1972; 3,418,055;Schwartzman; issued Dec. 24, 1968; and 3,410,645; Schwartzman; issued Nov. 12, 1968; all of which are herein incorporated by reference. Examples of applicators useful herein are commercially available from Dab-O-Matic, Mount Vernon, N.Y. Topical compositions of the present invention can also be delivered via any conventional hair care products, including, but not limited to shampoos, conditioners, styling products or other leave-in or rinse-off products.

### EXAMPLES

The following are non4imiting examples of the hair growth regulating compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified. As used herein, “minors” refers to those optional components such as preservatives, viscosity modifiers, pH modifiers, fragrances, foam boosters, and the like. As is apparent to one of ordinary skill in the art, the selection of these minors will vary depending on the physical and chemical characteristics of the particular ingredients selected to make the present invention as described herein.

Examples 1-8 are non-limiting examples of topical compositions used in the method of the present invention:

| Ingredient | 1 | 2 | 3 | 4 | 5 | 6 | 7 | 8 |
|---|---|---|---|---|---|---|---|---|
| zinc lactobionate | 10.0 | 5.0 | 5.0 | — | 6.0 | 0.1 | 1.0 | 15.0 |
| sodium lactobionate | — | — | 1.0 | 5.0 | — | — | — | — |
| zinc gluconate | — | 3.0 | — | — | — | — | 6.0 | — |
| minoxidil | — | 2.5 | — | — | 2.0 | 2.5 | — | — |
| ketoconazole | — | — | — | — | — | 2.0 | — | — |
| Tween 20 | 1.0 | — | 0.20 | — | — | — | — | — |
| propylene glycol | 10.0 | 8.0 | 15.0 | 20.0 | 25.0 | | 15.0 | 10.0 |
| dimethylisosorbide | 18.0 | 19.0 | — | 18.0 | 18.0 | — | — | — |
| C₁₂-C₁₅ alkyl octanoate | — | — | 19.0 | — | — | — | — | — |
| hydroxypropyl | — | — | 0.10 | — | — | 0.20 | — | — |
| cellulose | | | | | | | | |
| Silicone | — | — | — | 1.0 | — | 5.0 | — | 2.0 |
| polyquaternium 10 | — | — | — | 0.50 | 1.0 | — | — | — |
| ethyl alcohol and | 30.0 | q.s. to | q.s. to | — | — | q.s. to | 25.0 | 30.0 |
| minors | | 100 | 100 | | | 100 | | |
| water and minors | q.s. to | — | — | q.s. to | q.s. to | 10.0 | q.s. to | q.s. to |
| | 100 | | | 100 | 100 | | 100 | 100 |

Examples 1-8 may be prepared as follows:
1. Add the ethyl alcohol, if present, and water, if present, and polyquaternium 10, if present, into mixing container and mix for 1 to 2 hours.
2. Add the ethyl alcohol, if present, and Tween 20, if present, into mixing container and agitate until combined.
3. Add propylene glycol, if present, and dimethylisosobide, if present, and C12-C15 alkyl octanoate, if present, into mixing container and mix until in a clear solution.
4. Add the metal complex(es) of an oxidized carbohydrate and mix with a high shear mixer for 10 minutes.
5. Add hydroxypropyl cellulose, if present, to the mixture and mix with a standard mixer for 4 to 5 hours.
6. Add the remaining ingredients and mix an additional 10 minutes.

Examples 9-17 are non-limiting examples of topical compositions used in the method of the present invention:

| Ingredient | 9 | 10 | 11 | 12 | 13 | 14 | 15 | 16 | 17 |
|---|---|---|---|---|---|---|---|---|---|
| zinc lactobionate | 5.0 | — | — | — | — | 5.0 | — | — | 5.0 |
| zinc gluconate | 1.0 | — | — | 1.0 | — | — | — | 1.0 | — |
| copper lactobionate | — | 5.0 | — | — | — | — | — | — | — |
| lithium gluconate | — | — | 5.0 | — | — | — | — | — | — |
| copper galacturonate | — | — | — | 5.0 | — | — | — | — | — |
| zinc glucoheptonate | — | — | — | — | 5.0 | — | — | — | — |
| copper maltobionate | — | — | — | — | — | — | 5.0 | — | — |
| zinc alginate | — | — | — | — | — | — | — | 5.0 | — |
| Minoxidil | — | 2.5 | — | — | 2.0 | — | — | — | — |
| Finasteride | — | — | 1.0 | — | — | 1.0 | — | — | — |
| zinc pyrithione | 1.0 | — | — | — | — | — | — | — | 1.0 |
| Tween 20 | 1.0 | — | 0.20 | — | — | — | — | — | 1.0 |
| propylene glycol | 10.0 | 8.0 | 15.0 | 20.0 | 25.0 | 6.0 | 12.0 | 18.0 | 10.0 |
| dimethylisosorbide | 18.0 | 19.0 | — | 18.0 | 18.0 | — | — | — | 18.0 |
| C₁₂-C₁₅ alkyl octanoate | — | — | 19.0 | — | — | — | — | — | — |
| Hydroxypropyl | — | — | 0.10 | — | — | 0.5 | 0.1 | — | — |
| cellulose | | | | | | | | | |
| Polyquaterium 10 | — | — | 0.0 | 0.50 | 1.0 | — | 0.5 | — | — |
| ethyl alcohol and | 30.0 | q.s. to | q.s. to | — | 10.0 | — | q.s. to | — | 30.0 |
| minors | | 100 | 100 | | | | 100 | | |
| Water and minors | q.s. to | — | — | q.s. to | q.s. to | q.s. to | 10.0 | q.s. to | q.s. to |
| | 100 | | | 100 | 100 | 100 | | 100 | 100 |

Examples 9-17 may be prepared in the same manner as Examples 1-8.

Examples 18 and 19 are non-limiting examples of tablet compositions which can be used in the method of the present invention:

| Ingredient | Example 18 (mg) | Example 19 (mg) |
|---|---|---|
| Zinc lactobionate | 100 | 0.25 |
| Crospovidone | 15 | 0.0 |
| Lactose, hydrous | 200 | 0.0 |
| Microcrystalline cellulose | 80 | 0.0 |
| Magnesium stearate | 5 | 2.0 |
| Polyvinylpyrrolidone | 0.0 | 3.0 |
| Sodium starch glycolate | 0.0 | 2.0 |
| Dicalcium phosphate | 0.0 | 75.0 |
| Talc | 0.0 | 2.75 |
| Methanol | 0.0 | 20.0 |
| Starch 1500 | 0.0 | 15.0 |

Example 18 may be prepared as follows:
1. Add the zinc lactobionate, the crospovidone and the microcrystalline cellulose into a twin-shell blender and mix for 20 minutes.
2. Sieve the mixture through a 40 mesh screen and return to the twin-shell blender.
3. Add the lactose hydrous and mix for 25 minutes.
4. Add the magnesium stearate and mix for 5 minutes.
5. Compress into tablets on a standard rotary tablet press.
Example 19 may be prepared as follows:
1. Dissolve the zinc lactobionate and polyvinylpyrrolidone in the methanol under agitation.
2. Add the sodium starch glycolate, dicalcium phosphate, and starch 1500 into a high shear mixer and mix for 15 minutes.
3. Add the methanol solution to the high-shear blender over a 10 minute period and then mix for an additional 10 minutes until granules are formed.
4. Transfer the wetted mass into a fluid bed dryer and dry at 45° C. for 2 hours.
5. Sieve the dried granules through a 30 mesh screen and transfer back to the high-shear blender.
6. Add the talc and magnesium stearate and mix for 3 minutes.
7. Compress into tablets on a standard rotary tablet press

Example 20 is a non-limiting example of a composition which can be injected subcutaneously according to the method of the present invention.

| Ingredient | Example 20 (mg/mL) |
|---|---|
| Zinc lactobionate | 1.0 |
| Dibasic sodium phosphate | 7.0 |
| Monobasic sodium phosphate | 3.0 |
| Edetate disodium | 0.1 |
| Benzalkonium chloride | 0.1 |
| Water for injection | QS to 10 liters |

Example 20 may be prepared as follows:
1. The zinc lactobionate is micronized in a jet mill and sterilized by exposing it to 2.5 Mrad of radiation from a cobalt 60 source.
2. The dibasic sodium phosphate, monobasic sodium phosphate, edetate disodium, and benzalkonium chloride are dissolved in 9 liters of water for injection in a standard mixing tank.
3. The solution is filtered through a 0.22 micron filter to achieve sterilization.
4. The zinc lactobionate is added and mixed for 30 minutes under agitation.
5. The suspension is aseptically filled into 3 mL flint glass vials, stoppered and sealed on standard filling equipment.

### Examples 21 to 25 are non-limiting examples of shampoo compositions which embody the present invention.

| | Example Number | | | | |
|---|---|---|---|---|---|
| Ingredient | 21 | 22 | 23 | 24 | 25 |
| zinc lactobionate | 10.0 | 5.0 | 15.0 | — | 1.0 |
| copper maltobionate | — | — | — | 5.0 | — |
| zinc gluconate | — | 2.0 | — | — | — |
| ammonium laureth-3 sulfate | 14.00 | 10.0 | 14.0 | 12.0 | 12.50 |
| ammonium lauryl sulfate | — | 6.0 | — | 6.0 | — |
| Cocamidopropylbetaine | 2.70 | — | 2.70 | — | 4.20 |
| Polyquaternium-10 | 0.15 | — | — | 0.15 | — |
| cocamide MEA | 0.80 | 0.80 | 0.80 | 0.80 | — |
| cetyl alcohol | — | 0.42 | 0.42 | 0.42 | — |
| ethylene glycol distearate | 1.50 | 1.50 | 0.75 | 1.50 | 0.75 |
| zinc pyrithione | 1.0 | 1.0 | — | — | — |
| Dimethicone | 0.5 | 0.3 | 0.5 | 0.3 | 0.5 |

| | |
|---|---|
| water and minors | q.s. to 100% |

Examples 21 to 25 may be prepared according to any conventional method for making shampoos.

Examples 26 to 30 are non-limiting examples of products prepared processes, which embody the present invention.

| | Example Number | | | | |
|---|---|---|---|---|---|
| Ingredient | 26 | 27 | 28 | 29 | 30 |
| zinc sulphate | 3.8 | — | 3.8 | 3.8 | 3.8 |
| sodium hydroxide | 1.1 | — | 1.1 | 1.1 | — |
| zinc oxide | — | 1.1 | — | — | — |
| lactobionic acid | 9.5 | 9.5 | — | — | — |
| gluconic acid | — | — | 5.18 | — | — |
| maltobionic acid | — | — | — | 9.5 | — |
| sodium lactobionate | — | — | — | — | 10.0 |

| | |
|---|---|
| water and minors | q.s. to 100% |

Examples 26 to 30 may be prepared as follows:
1. Add the metal-salt and the carboxylic acid of carbohydrate and the carrier, in any order, agitate and heat, if necessary, to make homogeneous.
2. Adjust the pH of the result with sodium hydroxide, if desired. The resulting formulation will contain from about 0.001% to about 99.9%, by weight, of at least one metal complex of an oxidized carbohydrate, as desired.

What is claimed is:

1. A method for regulating the growth of hair comprising administering to a mammal, an effective amount of a composition comprising:
(a) from about 0.001% to about 99.9%, by weight, of at least one metal complex of an oxidized carbohydrate, wherein said metal complex of an oxidized carbohydrate is neither zinc gluconate nor manganese gluconate; and
(b) from about 0.1% to about 99.999%, by weight, of a vehicle.

2. The method of claim 1, wherein said composition comprises:
(a) from about 0.001% to about 15%, by weight, of said at least one metal complex of an oxidized carbohydrate; and
(b) from about 85% to about 99.999%, by weight, of said vehicle.

3. The method of claim 1, wherein the metal comprising said at least one metal complex is selected from the group consisting of lithium, sodium, silver, gold, zinc, copper, nickel, iron, chromium, calcium, magnesium, molybdenum, cobalt, palladium, platinum, tin, and mixtures thereof.

4. The method of claim 3, wherein said metal comprising at least one said metal complex is selected from the group consisting of lithium, sodium, zinc, copper, and mixtures thereof.

5. The method of claim 4, wherein said metal comprising said at least one metal complex is zinc.

6. The method of claim 1, wherein said oxidized carbohydrate is selected from the group consisting of oxidized aldoses, oxidized ketoses, oxidized trioses, oxidized tetroses, oxidized pentoses, oxidized hexoses, and mixtures thereof.

7. The method of claim 1, wherein said oxidized carbohydrate is selected from the group consisting of ribonic acid; ribulonic acid; arabinonic acid; xylonic acid; xylulonic acid; lyxonic acid; allonic acid; altronic acid; gluconic acid; mannonic acid; gulonic acid; idonic acid; galactonic acid; talonic acid; glucoheptonic acid; psiconic acid; fructonic acid; sorbonic acid; tagatonic acid; lactobionic acid; maltobionic acid; isomaltobionic acid; cellobionic acid; oxidized malto-oligosaccharide; oxidized cello-oligosaccharide; oxidized cellulose; chitin; gum arabic; gum karaya; gum xanthan; oxidized gum guar; oxidized locust bean gum; oxidized agars; oxidized algins; oxidized gellan gum; and mixtures thereof.

8. The method of claim 7, wherein said oxidized carbohydrate is selected from the group consisting of lactobionic acid; maltobionic acid; isomaltobionic acid; cellobionic acid; and mixtures thereof.

9. The method of claim 8, wherein said oxidized carbohydrate is lactobionic acid.

10. The method of claim 1, wherein said oxidized carbohydrate is not gluconate.

11. The method of claim 1, wherein said composition further comprises zinc gluconate, manganese gluconate, or both.

12. The method of claim 1, wherein the carbohydrate comprising said oxidized carbohydrate is selected from the group consisting of monosaccharides, disaccharides, polysaccharides, and mixtures thereof.

13. The method of claim 1, wherein said vehicle comprises at least one ingredient selected from the group consisting of solvents; thickeners; propellants; powders; fillers; plasticizers; lubricants; and emollients and humectants; and mixtures thereof.

14. The method of claim 13, wherein said solvent is selected from the group consisting of water, C₂ to C₃ monohydric alcohols, propylene glycol, and mixtures thereof.

15. The method of claim 14, wherein said vehicle comprises a solvent comprising:
(a) from about 50% to about 70%, by weight, of water;
(b) from about 20% to about 40%, by weight, of ethanol; and
(c) from about 5% to about 20%, by weight, of propylene glycol.

16. The method of claim 14, wherein said solvent comprises at least 5% propylene glycol.

17. The method of claim 21, wherein said solvent comprises from about 10% to about 15%, by weight, of propylene glycol.

18. The method of claim 1, wherein said composition further comprises at least one ingredient selected from the group consisting of surfactants, conditioning agents, cationic polymers, anti-dandruff actives, activity enhancers, penetration enhancers, dyes, suspending agents, non-steroidal anti-inflammatory drugs, topical anesthetics, sunscreen actives, flavoring agents, preservatives, sweeteners, and mixtures thereof.

19. The method of claim 18, wherein:
(a) said conditioning agents are selected from the group consisting of polydimethylsiloxane, polydiethylsiloxane, polymethylphenylsiloxane, amodimethicone, trimethylsilylamodimethicone, paraffin oil, mineral oil, polydecene, 1-decene homopolymer, C₈-C₁₀ triester of trimethylolpropane, polyethylene glycol, and mixtures thereof; and
(b) said cationic polymers selected from the group consisting of Polyquaternium 10, Polyquaternium 24, guar hydroxypropyltrimethylammonium chloride, Polyquaternium 16, and mixtures thereof; and
(c) said anti-dandruff actives selected from the group consisting of zinc pyrithione, selenium sulfide, sulfur, ketoconazole, climbazole, and mixtures thereof; and
(d) said activity enhancers selected from the group consisting of minoxidil, finasteride, cyclosporin, ketoconazole, triclocarbon, triclosan, zinc pyrithione, itraconazole, hinokitiol, mipirocin, hydrocortisone, tenidap, triiodothyrionine, latanoprost, isotretinoin, acitretin, tazarotene, nicotinic acid, niacinamide, glycosaminoglycanase inhibitors, ethylenediaminetetraacetic acid, oleanolic acid, ursolic acid, interleukin-1 inhibitors, interleukin-6 inhibitors, interleukin-10 promotors, saponins, triterpenes, betulinic acid, betulonic acid, crataegolic acid, celastrol, asiatic acid, inhibitors of 5-α-reductase, progesterone, 1,4-methyl4-azasteroids, 17-⊖-N,N-diethylcarbamoyl4-methyl4-aza-5-α-androstan-3-one, androgen receptor antagonists, cyproterone acetate, azelaic acid, diazoxide, potassium channel openers, cromakalin, phenytoin, dutasteride, coal tar, zinc gluconate, manganese gluconate, glucocortisoids, macrolides, aminexil, ginkgo biloba, ivy, methyl salicylate, clinacycin hydrochloride, benzoyl peroxide, benzyl peroxide, minocyclin, and mixtures thereof;
(e) said penetration enhancers selected from the group consisting of propan-2-ol; 1-propan-1,2-diol; propan-1-ol; di-isopropyl adipate; dimethylsulfonyl oxide; octonol;
niacinamide; and mixtures thereof; and
(f) said suspending agents selected from the group consisting of ethylene glycol monostearate, ethylene glycol distearate, stearyl stearate, cetyl palmitate, xanthan gum, copolymers of acrylic acid crosslinked with polyallylsucrose, methylcellulose, hydroxybutyl methylcellulose, hydroxypropylcellulose, hydroxypropyl methylcellulose, hydroxyethyl ethylcellulose, hydroxyethylcellulose, guar gum, polyvinyl alcohol, polyvinyl pyrrolidone, hydroxypropyl guar gum, starch, trihydroxystearin, and mixtures thereof.

20. The method of claim 19, wherein said activity enhancers are selected from the group consisting of minoxidil, finasteride, cyclosporin, ketoconazole, triclocarbon, triclosan, zinc pyrithione, itraconazole, tenidap, niacinamide, triterpenes, betulinic acid, betulonic acid, zinc gluconate, manganese gluconate, glucocortisoids, and mixtures thereof.

21. The method of claim 1, wherein said oxidized carbohydrate is amido-, amino-, or phospho-substituted.

22. The method of claim 9, wherein said lactobionic acid is sulfo-substituted.

23. The method of claim 1, wherein said composition is administered orally, parenterally, or topically.

24. The method of claim 23, wherein said composition is administered in a form selected from the group consisting of tablets, capsules, caplets, creams, gels, hydrogels, lotions, shampoos, rinses, tonics, sprays, ointments, mousses and pomades.

25. The method of claim 23, wherein said composition is administered topically.

26. The method of claim 1, wherein said composition is packaged as a component part of a kit, said kit containing at least one additional component for use in pre-treatment or post-treatment steps, said steps being part of a regimen for regulating hair growth performance.

27. A method for regulating the growth of hair comprising administering topically an effective amount of a composition comprising:
(a) zinc lactobionate;
(b) zinc gluconate, zinc pyrithione, or both; and
(c) a vehicle.

28. A method for regulating the growth of hair comprising administering to a mammal, an effective amount of a composition comprising:
(a) from about 0.001% to about 99.9%, by weight, of zinc lactobionate; and
(b) from about 0.1% to about 99.999%, by weight, of a vehicle.

29. A method for regulating the growth of hair comprising administering to a mammal, an effective amount of a composition comprising:
(a) from about 0.001% to about 99.9%, by weight, of at least one metal complex of an oxidized carbohydrate having a solubility in water of at least 10%; and
(b) from about 0.1% to about 99.999%, by weight, of a vehicle.

30. The method claim 29, wherein said at least one metal complex of an oxidized carbohydrate has a solubility in water of at least 40%.

31. The method of claim 30, wherein said at least one metal complex of an oxidized carbohydrate has a solubility in water of at least 50%.

32. The method of claim 31, wherein said at least one metal complex of an oxidized carbohydrate has a solubility in water of at least 60%.

33. The method of claim 1, wherein said at least one metal complex of an oxidized carbohydrate is zinc lactobionate.

34. A method for regulating the growth of hair comprising administering to a mammal, an effective amount of a composition that is the product of a process comprising the steps (a) to (c) in any order:
(a) adding from about 1% to about 25%, by weight, of a metal salt, and mixing;
(b) adding from about 1% to about 30%, by weight, of a carboxylic acid of a carbohydrate, and mixing; and
(c) adding from about 0.1% to about 99.999%, by weight, of a vehicle, and mixing;
wherein said product comprises from about 0.001% to about 99.9%, by weight, of at least one metal complex of an oxidized carbohydrate, wherein said metal complex of an oxidized carbohydrate is neither zinc gluconate nor manganese gluconate.

35. The method of claim 34, wherein the composition is the product of a process comprising the steps (a) to (c) in any order:
(d) adding from about 3% to about 10%, by weight, of a metal salt, and mixing;
(e) adding from about 5% to about 25%, by weight, of a carboxylic acid of a carbohydrate, and mixing; and
(f) adding from about 0.1% to about 99.999%, by weight, of a vehicle, and mixing;
wherein said product comprises from about 0.001% to about 15%, by weight, of said metal complex of an oxidized carbohydrate.

36. The method of claim 34, wherein the metal comprising said metal salt is selected from the group consisting of lithium, sodium, silver, gold, zinc, copper, nickel, iron, chromium, calcium, magnesium, molybdenum, cobalt, palladium, platinum, tin, and mixtures thereof.

37. The method of claim 36, wherein said metal comprising at least one said metal complex is selected from the group consisting of lithium, sodium, zinc, copper, and mixtures thereof.

38. The method of claim 37, wherein said metal comprising said at least one metal complex is zinc.

39. The method of claim 34, wherein said carboxylic acid of a carbohydrate is selected from the group consisting of lactobionic acid; maltobionic acid; isomaltobionic acid; cellobionic acid; and mixtures thereof.

40. The method of claim 39, wherein said carboxylic acid of a carbohydrate is lactobionic acid.

41. The method of claim 34, wherein the salt portion of said metal salt is selected from the group consisting of chlorides, sulfates, acetates, oxides, and mixtures thereof.

42. The method of claim 34, wherein said metal salt is selected from the group consisting of zinc sulfate, zinc acetate, zinc oxide, cupric chloride, cupric sulfate, cupric acetate, and copper oxide.

43. The method of claim 42, wherein said metal salt is zinc sulfate.

44. The method of claim 40, wherein said metal salt is zinc sulfate.
